SEQUENCE LISTING (29 sequences)

SEQ ID NO: 1 - DNA, 44 bp, Artificial Sequence
  Synthetic oligonucleotide referred to as an adaptor
acttcagaaccgcaatgcacgatacgtctcgggaacgctgaaga

SEQ ID NO: 2 - DNA, 56 bp, Artificial Sequence
  Synthetic oligonucleotide referred to as an adaptor
ggctccagcggctaacgatagctcgagctcgagcaatgacgtctcgactcagcaga

SEQ ID NO: 3 - DNA, 56 bp, Artificial Sequence
  Synthetic oligonucleotide referred to as an adaptor
actgctgacgtactgcgagaagctcgagctcgagcgactgcgcttcgactggagac

SEQ ID NO: 4 - DNA, 68 bp, Artificial Sequence
  Synthetic oligonucleotide referred to as an adaptor
aagtcggaggccaagcggtcttaggaagacaagctcgagctcgagcgatcgggccgtacgtccaactt

SEQ ID NO: 5 - DNA, 14 bp, Artificial Sequence
  Synthetic oligonucleotide referred to as an adaptor
gctcgagctcgagc

SEQ ID NO: 6 - DNA, 20 bp, Artificial Sequence
  Stabilizing sequence of a synthetic oligonucleotide referred to as an adaptor
gctcgagtgttgtctcgagc

SEQ ID NO: 7 - DNA, 56 bp, Artificial Sequence
  Synthetic oligonucleotide referred to as an adaptor
gctccagcggctaacgatgctcgagctcgagcaatgacgtctcgactcagcagann

SEQ ID NO: 8 - DNA, 57 bp, Artificial Sequence
  Synthetic oligonucleotide referred to as an adaptor
tctccagtcgaagcgcagtcgctcgagctcgagcttctcgcagtacgtcagcagtnn

SEQ ID NO: 9 - DNA, 67 bp, Artificial Sequence
  Synthetic oligonucleotide referred to as an adaptor
agtcggaggccaagcggtcttaggaagacaagctcgagctcgagcgatcgggccgtacgtccaactt

SEQ ID NO: 10 - DNA, 22 bp, Artificial Sequence
  Stabilizing sequence of a synthetic oligonucleotide referred to as an adaptor
agacaagctcgagctcgagcga

SEQ ID NO: 11 - DNA, 33 bp, Artificial Sequence
  Stabilizing sequence of a synthetic oligonucleotide referred to as an adaptor
agacaacaagatcgagctcgatcttgactcctg

SEQ ID NO: 12 - DNA, 33 bp, Artificial Sequence
  Stabilizing sequence of a synthetic oligonucleotide referred to as an adaptor
agacaacacggtcgagctcgaccgtgactcctg

SEQ ID NO: 13 - DNA, 37 bp, Artificial Sequence
  Stabilizing sequence of a synthetic oligonucleotide referred to as an adaptor
agacaacagaagatcgagctcgatcttctgactcctg

SEQ ID NO: 14 - DNA, 37 bp, Artificial Sequence
  Stabilizing sequence of a synthetic oligonucleotide referred to as an adaptor
agacaaccgacggtcgagctcgaccgtcggactcctg

SEQ ID NO: 15 - DNA, 21 bp, Artificial Sequence
  Stabilizing sequence of a synthetic oligonucleotide referred to as an adaptor
agacaacgagctgcactcctg

SEQ ID NO: 16 - DNA, 15 bp, Artificial Sequence
  Probe
acucuagcugacuag

SEQ ID NO: 17 - DNA, 35 bp, Artificial Sequence
  Target sequence
gagtnnnnnnnnnnnnnnnntgagatcgactgatc

SEQ ID NO: 18 - DNA, 12 bp, Artificial Sequence
  Probe
aatgtgatacca

SEQ ID NO: 19 - DNA, 12 bp, Artificial Sequence
  Probe
tgctcctctagg

SEQ ID NO: 20 - DNA, 12 bp, Artificial Sequence
  Probe
gcaagtgactac

SEQ ID NO: 21 - DNA, 12 bp, Artificial Sequence
  Probe
ctgcaacgggtt

SEQ ID NO: 22 - DNA, 12 bp, Artificial Sequence
  Portion of target amplicon
aaaaaaaaaaaa

SEQ ID NO: 23 - DNA, 12 bp, Artificial Sequence
  Portion of target amplicon
tttttttttttt

SEQ ID NO: 24 - DNA, 12 bp, Artificial Sequence
  Portion of target amplicon
cccccccccccc

SEQ ID NO: 25 - DNA, 12 bp, Artificial Sequence
  Portion of target amplicon
gggggggggggg

SEQ ID NO: 26 - DNA, 47 bp, Artificial Sequence
  Target sequence
nnnnnnnnnnnnnnnnnnnngatcatcgtcagcagtcgcgtagctag

SEQ ID NO: 27 - DNA, 28 bp, Artificial Sequence
  Probe
nnnnctagtagcagtcgtcagcgcatcg

SEQ ID NO: 28 - DNA, 39 bp, Artificial Sequence
  Probe
nnnncnnnnnnnctagtagcagtcgtcagcgcatcgatc

SEQ ID NO: 29 - DNA, 47 bp, Artificial Sequence
  Target sequence
nnnnnnnnnnnngnnnnnnngatcatcgtcagcagtcgcgtagctag

(19) United States
(12) Patent Application Publication
(10) Pub. No.: US 2009/0263872 A1
(43) Pub. Date: Oct. 22, 2009

(54) Methods and compositions for preventing bias in amplification and sequencing reactions

(75) Inventors: Karen Shannon, Los Gatos, CA (US); Matthew J. Callow, Redwood City, CA (US); Andrew Sparks, Los Gatos, CA (US); Arnold Oliphant, Sunnyvale, CA (US)

Correspondence Address: MORGAN, LEWIS & BOCKIUS, LLP, ONE MARKET SPEAR STREET TOWER, SAN FRANCISCO, CA, 94105, US

(73) Assignee: COMPLETE GENOMICS INC., Mountain View, CA (US)

(21) Appl. No.: 12/359,165

(22) Filed: Jan. 23, 2009

Related U.S. Application Data
(60) Provisional application No. 61/023,010, filed on Jan. 23, 2008.
(60) Provisional application No. 61/023,247, filed on Jan. 24, 2008.

(51) Int. Cl. C12P 19/34; C12N 15/11

(52) U.S. Cl. 435/91.4; 536/23.1

(57) ABSTRACT

The present invention is directed to compositions and methods for nucleic acid identification and detection. Compositions and methods of the present invention include template nucleic acids with stabilizing sequences. The present invention also includes concatemers formed from template nucleic acids that have stabilizing sequences, arrays of such concatemers, as well as methods for identifying and detecting sequences of such concatemers.

### CROSS REFERENCE TO RELATED APPLICATIONS

This application claims the benefit of priority to U.S. Provisional Patent Application Nos. 61/023,010, filed Jan. 23, 2008 and 61/023,247, filed Jan. 24, 2008, each of which is hereby incorporated by reference in its entirety.

### BACKGROUND OF THE INVENTION

Large-scale genomic sequence analysis is a key step toward understanding a wide range of biological phenomena. The need for low-cost, high-throughput sequencing and re-sequencing has led to the development of new approaches to sequencing that employ parallel analysis of multiple nucleic acid targets simultaneously.

Conventional methods of sequencing are generally restricted to determining a few tens of nucleotides before signals become significantly degraded, thus placing a significant limit on overall sequencing efficiency. Conventional methods of sequencing are also often limited by signal-to-noise ratios that render such methods unsuitable for single-molecule sequencing.

It would be advantageous for the field if methods and compositions could be designed to increase the efficiency of sequencing reactions.

### SUMMARY OF THE INVENTION

Accordingly, the present invention provides methods and compositions for sequencing reactions.

In one aspect, the present invention provides a method for synthesizing nucleic acid amplicons with enhanced stability. This method includes the steps of (1) providing a target nucleic acid; (2) ligating a first arm of a first adaptor to one end of the target nucleic acid and a second arm of the first adaptor to the other end of the target nucleic acid, to form a first linear construct. In a further aspect, the first adaptor comprises a recognition site for a first type IIs restriction endonuclease. In this aspect, the method further comprises the steps of amplifying the first linear construct with primers comprising one or more stabilizing sequences to produce amplification products and circularizing the amplification products to form circular templates. This method also includes amplifying the circular templates using a rolling circle replication method to form nucleic acid amplicons.

In one aspect, the present invention provides compositions comprising a nucleotide sequence according to at least one of SEQ ID NOs: 1-4.

In one aspect, the invention provides a composition that comprises a substrate with a surface. The surface of the substrate in turn comprises a plurality of concatemers immobilized on the surface. In a further aspect, each monomer of each of the plurality of concatemers comprises: (1) a first adaptor that comprises a nucleotide sequence according to SEQ ID NO: 1; (2) a second adaptor that comprises a nucleotide sequence according to SEQ ID NO: 2; (3) a third adaptor that comprises a nucleotide sequence according to SEQ ID NO: 3; (4) a fourth adaptor that comprises a nucleotide sequence according to SEQ ID NO: 4; (5) a first target sequence adjacent to the first adaptor; (6) a second target sequence adjacent to the second adaptor; (7) a third target sequence adjacent to the third adaptor; and (8) a fourth target sequence adjacent to the fourth adaptor.

### BRIEF DESCRIPTION OF THE DRAWINGS

FIG. 1 provides some exemplary embodiments of adaptor sequences of the invention.

FIG. 2 provides some exemplary embodiments of adaptor sequences of the invention (A) as well as exemplary components of adaptors of the invention (B).

FIG. 3 is an illustration of an exemplary sequencing method of the invention.

FIG. 4 is an illustration of an exemplary method for constructing nucleic acid templates of the invention.

FIG. 5 is an illustration of an exemplary method of forming concatemers of the invention.

FIG. 6 is an illustration of an exemplary method of forming nucleic acid templates of the invention.

FIG. 7 is an illustration of an exemplary method of forming nucleic acid templates of the invention.

FIG. 8 is an illustration of a four probe model system for assessing amplicon quantity and/or quality using methods of the invention.

FIG. 9 is an illustration of a four probe model system for assessing amplicon quantity and/or quality using engineered sequences downstream of each adaptor using methods of the invention.

FIG. 10 is an illustration of an exemplary method of sequencing of the invention.

FIG. 11 is an illustration of an exemplary method of forming amplicons of the invention.

FIG. 12 is a plot of the distribution of amplicons created from sequencing constructs as assessed by an assay of the invention.

FIG. 13 is a chart showing characteristics of exemplary stabilizing sequences inserted into adaptors, along with a graph showing the average fraction of color purity of amplicons containing adaptors with these stabilizing sequences as measured in a model system.

FIG. 14 is a plot of the distribution of amplicons created from sequencing constructs having engineered poly-nucleotide repeats as assessed by an assay of the invention.

FIG. 15 is a graph of the rate of amplicon production for four constructs each comprising a poly-nucleotide repeat.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### I. Overview

The present invention is directed to compositions and methods for nucleic acid identification and detection, which find use in a wide variety of applications as described herein.

The method for nucleic acid identification and detection using compositions and methods of the present invention includes extracting and fragmenting target nucleic acids from a sample. These fragmented nucleic acids are used to produce target nucleic acid templates that generally include one or more adaptors. The target nucleic acid templates are subjected to amplification methods to form nucleic acid concatemers, also referred to herein as nucleic acid “nanoballs” and “amplicons”. In some situations, these nanoballs are disposed on a surface. Sequencing applications are performed on the nucleic acid nanoballs of the invention, usually through sequencing by ligation techniques, including combinatorial probe anchor ligation (“cPAL”) methods, which are described in further detail below.

The target nucleic acid templates of the present invention generally include stabilizing sequences. In some cases, these stabilizing sequences are palindromic sequences. In some cases, target nucleic acid templates comprise at least two stabilizing sequences that are complementary to one another. When a concatemer is generated from target nucleic acid templates including such stabilizing sequences, the complementary sequences will hybridize to each other, thus enhancing intramolecular interaction of the concatemer and helping to prevent intermolecular interactions between different concatemers. Similarly, stabilizing sequences comprising palindromic sequences will in part direct the secondary structure conformation of concatemers generated from target nucleic acid templates comprising these sequences. In many cases, concatemers comprising stabilizing sequences according to the present invention will form more compact spherical shapes that occupy a smaller area when disposed on a surface than concatemers that do not contain such stabilizing sequences.

Target nucleic acid templates of the invention generally include one or more adaptors. These adaptors often include one or more functional elements, including stabilizing sequences such as those discussed above and described in further detail herein. These adaptors can also include one or more binding regions for initiation of biochemical reactions, such as sequencing reactions (through binding of an anchor probe) and circle dependent replication reactions (through binding of a replication primer). These binding regions are generally located in a region of the adaptor that is separated by at least one nucleotide from a region comprising a stabilizing sequence. This separation of the binding region from the stabilizing sequence can prevent secondary structure of a concatemer generated from target nucleic acid templates of the invention from impeding the binding region, thus keeping the binding region accessible to primers and/or enzymes for initiation of sequencing and/or amplification reactions.

As will be discussed in further detail below, target nucleic acid templates of the invention are generally circular single stranded nucleic acid molecules comprising target sequence interspersed with one or more adaptors. These circular templates are generally formed in a process that begins with double stranded nucleic acids that are processed according to methods described further herein to incorporate one or more adaptors into their linear sequence. As discussed above, these adaptors can comprise multiple functional elements, including stabilizing sequences and binding regions for sequencing and amplification reactions. These adaptors can also include recognition sites for restriction endonucleases, including Type IIs and Type III endonucleases. As will be described in more detail below, such recognition sites can play a key role in the construction of target nucleic acid templates of the invention containing multiple interspersed adaptors.

The target nucleic acid templates of the invention are used to produce concatemers that possess a secondary structure that can at least in part be directed by the sequence of the adaptors, particularly stabilizing sequences that those adaptors may contain. Adaptors can be designed according to methods described herein to improve the efficiency of both amplification and sequencing reactions, often through the way they direct the secondary structure of the concatemers. In some cases, adaptors described herein can prevent bias in amplification and sequencing reactions.

Concatemers are generally produced by conducting circle dependent replication reactions on target nucleic acid templates of the invention. Such circle dependent replication reactions generally include rolling circle replication methods utilizing polymerases such as phi29. In some cases, concatemers are generated from two or more primer sites simultaneously, such that a multi-strand concatemer is formed. When a multi-strand concatemer is formed form a target nucleic acid template comprising stabilizing sequences, the stabilizing sequences of the multiple strands can interact to produce a nucleic acid nanoball that has a tighter, more compressed or compact structure than would be seen with a nucleic acid nanoball comprising the same target sequences without any stabilizing sequences.

Concatemers produced as discussed above and described in further detail below can be used in a variety of sequencing reactions known in the art and described in further detail below. In some cases, concatemers are sequenced using a combinatorial probe-anchor ligation (cPAL) sequencing method that is described in further detail below.

### II. Compositions of the Invention

Compositions of the invention include nucleic acid templates, concatemers generated from such nucleic acid templates, as well as substrates comprising a surface with a plurality of such concatemers disposed on that surface.

In one aspect, the present invention provides nucleic acid templates comprising target nucleic acids and multiple interspersed adaptors, also referred to herein as “library constructs,” “circular templates”, “circular constructs”, “target nucleic acid templates”, and other grammatical equivalents. The nucleic acid template constructs of the invention are assembled by inserting adaptors molecules at a multiplicity of sites throughout each target nucleic acid. The interspersed adaptors permit acquisition of sequence information from multiple sites in the target nucleic acid consecutively or simultaneously.

The term “target nucleic acid” refers to a nucleic acid of interest. In one aspect, target nucleic acids of the invention are genomic nucleic acids, although other target nucleic acids can be used, including mRNA (and corresponding cDNAs, etc.). Target nucleic acids include naturally occurring or genetically altered or synthetically prepared nucleic acids (such as genomic DNA from a mammalian disease model). Target nucleic acids can be obtained from virtually any source and can be prepared using methods known in the art. For example, target nucleic acids can be directly isolated without amplification, isolated by amplification using methods known in the art, including without limitation polymerase chain reaction (PCR), strand displacement amplification (SDA), multiple displacement amplification (MDA), rolling circle amplification (RCA), rolling circle amplification (RCR) and other amplification (including whole genome amplification) methodologies. Target nucleic acids may also be obtained through cloning, including but not limited to cloning into vehicles such as plasmids, yeast, and bacterial artificial chromosomes.

In some aspects, the target nucleic acids comprise mRNAs or cDNAs. In certain embodiments, the target DNA is created using isolated transcripts from a biological sample. Isolated mRNA may be reverse transcribed into cDNAs using conventional techniques, again as described in Genome Analysis: A Laboratory Manual Series (Vols. I-IV) or Molecular Cloning: A Laboratory Manual.

Target nucleic acids can be obtained from a sample using methods known in the art. As will be appreciated, the sample may comprise any number of substances, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is generally described in PCT/US99/01705, such as PCR amplification reaction); purified samples, such as purified genomic DNA, RNA, proteins, etc.; raw samples (bacteria, virus, genomic DNA, etc.); as will be appreciated by those in the art, virtually any experimental manipulation may have been done on the sample. In one aspect, the nucleic acid constructs of the invention are formed from genomic DNA. In certain embodiments, the genomic DNA is obtained from whole blood or cell preparations from blood or cell cultures.

In an exemplary embodiment, genomic DNA is isolated from a target organism. By “target organism” is meant an organism of interest and as will be appreciated, this term encompasses any organism from which nucleic acids can be obtained, particularly from mammals, including humans, although in some embodiments, the target organism is a pathogen (for example for the detection of bacterial or viral infections). Methods of obtaining nucleic acids from target organisms are well known in the art. Samples comprising genomic DNA of humans find use in many embodiments. In some aspects such as whole genome sequencing, about 20 to about 1,000,0000 or more genome-equivalents of DNA are preferably obtained to ensure that the population of target DNA fragments sufficiently covers the entire genome. The number of genome equivalents obtained may depend in part on the methods used to further prepare fragments of the genomic DNA for use in accordance with the present invention.

The target nucleic acids used to make templates of the invention may be single stranded or double stranded, as specified, or contain portions of both double stranded or single stranded sequence. Depending on the application, the nucleic acids may be DNA (including genomic and cDNA), RNA (including mRNA and rRNA) or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

By “nucleic acid” or “oligonucleotide” or “polynucleotide” or grammatical equivalents herein means at least two nucleotides covalently linked together. A nucleic acid of the present invention will generally contain phosphodiester bonds, although in some cases, as outlined below (for example in the construction of primers and probes such as label probes), nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid (also referred to herein as “PNA”) backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with bicyclic structures including locked nucleic acids (also referred to herein as “LNA”), Koshkin et al., J. Am. Chem. Soc. 120:13252 3 (1998); positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169 176). Several nucleic acid analogs are described in Rawls, C & E News Jun. 2, 1997 page 35. “Locked nucleic acids” (LNA™) are also included within the definition of nucleic acid analogs. LNAs are a class of nucleic acid analogues in which the ribose ring is “locked” by a methylene bridge connecting the 2′-O atom with the 4′-C atom. All of these references are hereby expressly incorporated by reference in their entirety for all purposes and in particular for all teachings related to nucleic acids. These modifications of the ribose-phosphate backbone may be done to increase the stability and half-life of such molecules in physiological environments. For example, PNA:DNA and LNA-DNA hybrids can exhibit higher stability and thus may be used in some embodiments.

The nucleic acid templates of the invention comprise target nucleic acids and adaptors. As used herein, the term “adaptor” refers to an oligonucleotide of known sequence. Adaptors of use in the present invention may include a number of elements. The types and numbers of elements (also referred to herein as “features”, “functional elements” and grammatical equivalents) included in an adaptor will depend on the intended use of the adaptor. Adaptors of use in the present invention will generally include without limitation sites for restriction endonuclease recognition and/or cutting, particularly Type IIs recognition sites that allow for endonuclease binding at a recognition site within the adaptor and cutting outside the adaptor as described below, sites for primer binding (for amplifying the nucleic acid constructs) or anchor primer (sometimes also referred to herein as “anchor probes”) binding (for sequencing the target nucleic acids in the nucleic acid constructs), nickase sites, and the like. In some embodiments, adaptors will comprise a single recognition site for a restriction endonuclease, whereas in other embodiments, adaptors will comprise two or more recognition sites for one or more restriction endonucleases. As outlined herein, the recognition sites are frequently (but not exclusively) found at the termini of the adaptors, to allow cleavage of the double stranded constructs at the farthest possible position from the end of the adaptor. Adaptors of use in the invention are described herein and in U.S. application Ser. Nos. 12/265,593; 12/266,385; 11/938,106; 11/938,096; 11/982,467; 11/981,804; 11/981,797; 11/981,793; 11/981,767; 11/981,761; 11/981,730; 11/981,685; 11/981,661; 11/981,607; 11/981,605; 11/927,388; 11/927,356; 11/679,124; 11/541,225; 10/547,214; 11/451,691; 12/329,365; and 12/335,188, all of which are hereby incorporated by reference in their entirety, and particularly for all disclosure related to adaptors and target nucleic acid templates comprising adaptors.

In some embodiments, adaptors of the invention have a length of about 10 to about 250 nucleotides, depending on the number and size of the features included in the adaptors. In certain embodiments, adaptors of the invention have a length of about 50 nucleotides. In further embodiments, adaptors of use in the present invention have a length of about 20 to about 225, about 30 to about 200, about 40 to about 175, about 50 to about 150, about 60 to about 125, about 70 to about 100, and about 80 to about 90 nucleotides.

In further embodiments, adaptors may optionally include elements such that they can be ligated to a target nucleic acid as two “arms”. One or both of these arms may comprise an intact recognition site for a restriction endonuclease, or both arms may comprise part of a recognition site for a restriction endonuclease. In the latter case, circularization of a construct comprising a target nucleic acid bounded at each termini by an adaptor arm will reconstitute the entire recognition site.

In still further embodiments, adaptors of use in the invention will comprise different anchor binding sites (also referred to herein as “anchor sites”) at their 5′ and the 3′ ends. As described further herein, such anchor binding sites can be used in sequencing applications, including the combinatorial probe anchor ligation (cPAL) method of sequencing, described herein and in U.S. application Ser. Nos. 12/265,593; 12/266,385; 11/938,106; 11/938,096; 11/982,467; 11/981,804; 11/981,797; 11/981,793; 11/981,767; 11/981,761; 11/981,730; 11/981,685; 11/981,661; 11/981,607; 11/981,605; 11/927,388; 11/927,356; 11/679,124; 11/541,225; 10/547,214; 11/451,691; 12/329,365; and 12/335,188, all of which are hereby incorporated by reference in their entirety, and particularly for all disclosure related to sequencing by ligation.

In one aspect, adaptors of the invention are interspersed adaptors. By “interspersed adaptors” is meant herein oligonucleotides that are inserted at spaced locations within the interior region of a target nucleic acid. In one aspect, “interior” in reference to a target nucleic acid means a site internal to a target nucleic acid prior to processing, such as circularization and cleavage, that may introduce sequence inversions, or like transformations, which disrupt the ordering of nucleotides within a target nucleic acid. “Interspersed adaptors” can be inserted such that they interrupt a contiguous target sequence, thus conferring a spatial and distance orientation between the target sequences. That is, as outlined herein and in the incorporated applications, using endonucleases that cut outside of the recognition sequence allows the precise insertion (via ligation) of adaptors at defined intervals within the target sequence. This facilitates sequence reconstruction and alignment, as sequence runs of 10 bases each from a single adaptor can allow 20, 30, 40, etc. bases to be read without alignment, per se.

The nucleic acid template constructs of the invention contain multiple interspersed adaptors inserted into a target nucleic acid, and in a particular orientation. As discussed further herein, the target nucleic acids are produced from nucleic acids isolated from one or more cells, including one to several million cells. These nucleic acids are then fragmented using mechanical or enzymatic methods.

The target nucleic acid that becomes part of a nucleic acid template construct of the invention may have interspersed adaptors inserted at intervals within a contiguous region of the target nucleic acids at predetermined positions. The intervals may or may not be equal. In some aspects, the accuracy of the spacing between interspersed adaptors may be known only to an accuracy of one to a few nucleotides. In other aspects, the spacing of the adaptors is known, and the orientation of each adaptor relative to other adaptors in the library constructs is known. That is, in many embodiments, the adaptors are inserted at known distances, such that the target sequence on one terminus is contiguous in the naturally occurring genomic sequence with the target sequence on the other terminus. For example, in the case of a Type IIs restriction endonuclease that cuts 16 bases from the recognition site, if the recognition site is located 3 bases into the adaptor, the endonuclease cuts 13 bases from the end of the adaptor. Upon the insertion of a second adaptor, the target sequence “upstream” of the adaptor and the target sequence “downstream” of the adaptor are actually contiguous sequences in the original target sequence. Thus, the interspersed adaptors of the present invention are truly “inserted” into a target sequence rather than simply appended to the ends of fragments randomly generated through enzymatic and mechanical methods.

Although the embodiments of the invention described herein are generally described in terms of circular nucleic acid template constructs, it will be appreciated that nucleic acid template constructs may also be linear. Furthermore, nucleic acid template constructs of the invention may be single- or double-stranded, with the latter being preferred in some embodiments.

In further embodiments, nucleic acid templates formed from a plurality of genomic fragments can be used to create a library of nucleic acid templates. Such libraries of nucleic acid templates will in some embodiments encompass target nucleic acids that together encompass all or part of an entire genome. That is, by using a sufficient number of starting genomes (e.g. cells), combined with random fragmentation, the resulting target nucleic acids of a particular size that are used to create the circular templates of the invention sufficiently “cover” the genome, although as will be appreciated, on occasion, bias may be introduced inadvertently to prevent the entire genome from being represented.

The nucleic acid template constructs of the invention comprise multiple interspersed adaptors, and in some aspects, these interspersed adaptors comprise one or more recognition sites for restriction endonucleases. In further aspect, the adaptors comprise recognition sites for Type IIs endonucleases. Type-IIs endonucleases are generally commercially available and are well known in the art. Like their Type-II counterparts, Type-IIs endonucleases recognize specific sequences of nucleotide base pairs within a double stranded polynucleotide sequence. Upon recognizing that sequence, the endonuclease will cleave the polynucleotide sequence, generally leaving an overhang of one strand of the sequence, or “sticky end.” Type-IIs endonucleases also generally cleave outside of their recognition sites; the distance may be anywhere from about 2 to 30 nucleotides away from the recognition site depending on the particular endonuclease. Some Type-IIs endonucleases are “exact cutters” that cut a known number of bases away from their recognition sites. In some embodiments, Type IIs endonucleases are used that are not “exact cutters” but rather cut within a particular range (e.g. 6 to 8 nucleotides). Generally, Type IIs restriction endonucleases of use in the present invention have cleavage sites that are separated from their recognition sites by at least six nucleotides (i.e. the number of nucleotides between the end of the recognition site and the closest cleavage point). Exemplary Type IIs restriction endonucleases include, but are not limited to, Eco57M I, Mme I, Acu I, Bpm I, BceA I, Bbv I, BciV I, BpuE I, BseM II, BseR I, Bsg I, BsmF I, BtgZ I, Eci I, EcoP15 I, Eco57M I, Fok I, Hga I, Hph I, Mbo II, Mnl I, SfaN I, TspDT I, TspDW I, Taq II, and the like. In some exemplary embodiments, the Type IIs restriction endonucleases used in the present invention are AcuI, which has a cut length of about 16 bases with a 2-base 3′ overhang and EcoP15, which has a cut length of about 25 bases with a 2-base 5′ overhang. As will be discussed further below, the inclusion of a Type IIs site in the adaptors of the nucleic acid template constructs of the invention provides a tool for inserting multiple adaptors in a target nucleic acid at a defined location.

As will be appreciated, adaptors may also comprise other elements, including recognition sites for other (non-Type IIs) restriction endonucleases, including Type I and Type III restriction endonucleases, as well as Type II endonucleases (including IIB, IIE, IIG, IIM, and any other enzymes known in the art), primer binding sites for amplification as well as binding sites for probes used in sequencing reactions (“anchor probes”), described further herein. Type III endonucleases, similar to the Type IIs endonucleases, cut at sites outside of their recognition sites. These enzymes, as for many of the enzymes recited herein, may also be used in to control the inactivation and activation of restriction endonuclease recognition sites through methylation, as described in U.S. application Ser. Nos. 12/265,593; 12/266,385; 12/329,365; and 12/335,188, each of which is herein incorporated by reference in its entirety for all purposes and in particular for all teachings related to the insertion of multiple adaptors and the control over recognition sites for restriction endonucleases contained in such adaptors.

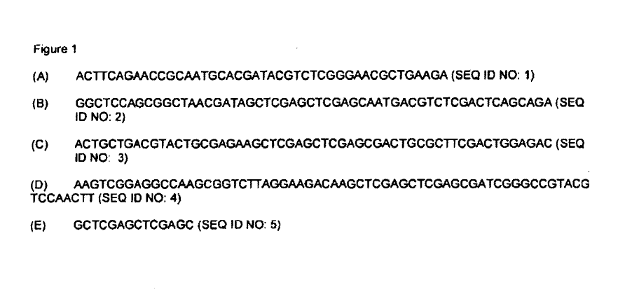

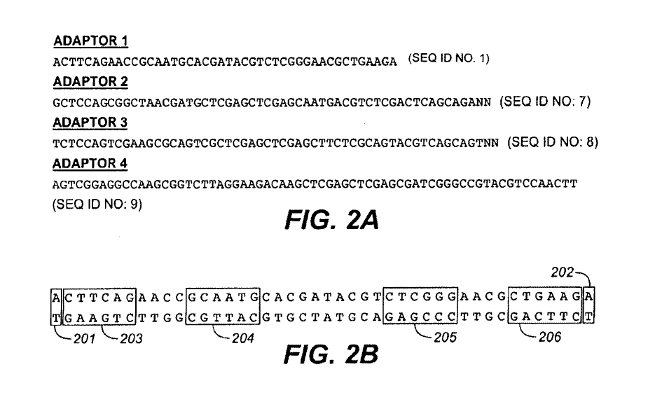

In one aspect, adaptors of use in the invention have sequences as shown in FIGS. 1 and 2 (SEQ ID NOs. 1-9). In further aspects, adaptors of use in the invention may comprise one or more of the sequences illustrated in FIGS. 1 and 2. As will be appreciated, sequences that have at least 65%, 70%, 75%, 80%, 85%, 90%, 91%, 92%, 93%, 94%, 95%, 96%, 97%, 98%, and 99% sequence identity to the sequences provided in FIGS. 1 and 2 are also encompassed by the present invention. As identified in the schematic of one of the adaptors in FIG. 2B, adaptors can comprise multiple functional features, including recognition sites for Type IIs restriction endonucleases (203 and 206), sites for nicking endonucleases (204) as well as sequences that can influence secondary characteristics, such as bases to disrupt hairpins (201 and 202).

In further embodiments, adaptors of use in the invention contain stabilizing sequences. By the term “stabilizing sequences” or “stabilization sequences” herein is meant nucleic acid sequences that facilitate DNB formation and/or stability. For example, stabilization sequences can allow the formation of secondary structures within the DNBs of the invention. Complementary sequences, including palindromic sequences, find particular use in the invention. In some cases, it is possible to use nucleic acid binding proteins and their recognition sequences as stabilization sequences, or crosslinking components as is more fully described below. Multiple configurations of stabilizing sequences can be used in the invention, and will depend in part upon the numbers of adaptors used in the constructs, the desired structures of the amplicon, and the placement of the binding region in each construct relative to the stabilizing sequences.

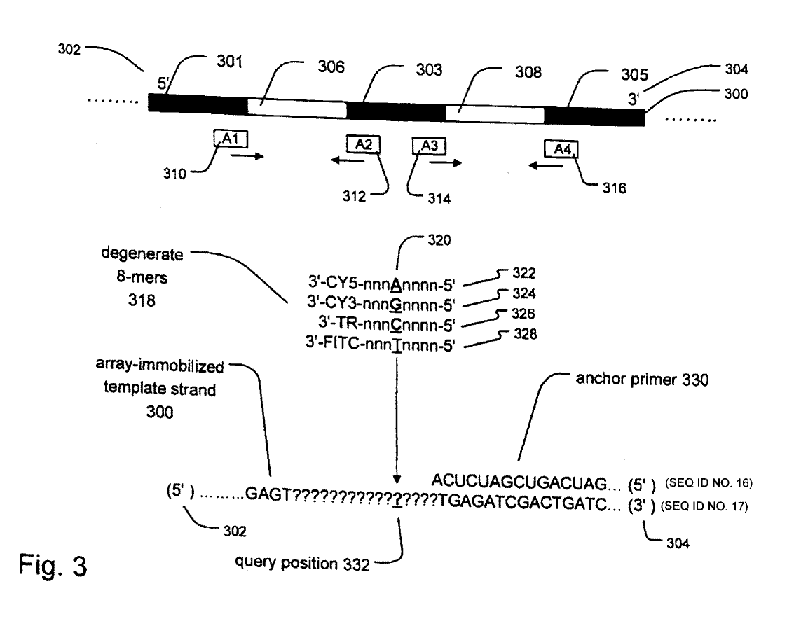

A number of potential configurations of stabilizing sequence-containing adaptors in library constructs are illustrated in FIG. 3. For example, library construct 310 comprises target nucleic acid 301 and adaptors 302 having stabilizing sequences, as represented by the arrows within the adaptors 302. Stabilizing sequences are generally nucleic acid sequences in the library constructs that promote intramolecular bonding and/or folding of the nucleic acid amplicons. Such stabilizing sequences may be palindromic sequences, complementary sequences, sequences that are amenable to cross-linking and the like and combinations thereof. For example, the stabilizing sequence in each adaptor 302 may be a palindromic sequence such as GCTCGAGCTCGAGC (SEQ ID NO. 5) contained within a single adaptor as indicated in library construct 310. Alternatively, as indicated in library construct 320, the stabilizing sequences in the adaptors may be half of a palindromic sequence, e.g., GCTCGAG in one adaptor 304, the complementary sequence CTCGAGC in the next adaptor 405, GCTCGAG in the third adaptor 304, CTCGAGC in the fourth adaptor 305 and so on. Library construct 330 shows yet another alternative, where an entire palindromic sequence is contained in a single adaptor 302; however, some adaptors 306 do not contain any stabilizing sequences.

Yet another alternative is shown in library construct 340, where the stabilizing sequences in the adaptors 304 and 305 are, e.g., half of a palindromic sequence; however, only every other adaptor comprises a stabilizing sequence. Library construct 350 shows yet another alternative, where an entire stabilizing sequence is contained in a single adaptor 302; however, two out of three adaptors 306 do not contain any stabilizing sequences. Library construct 360 comprises adaptors 304 and 305 comprising, e.g., half of a palindromic sequence; however, only every third adaptor comprises a stabilizing sequence. In the library constructs, as demonstrated, every adaptor may comprise a stabilizing sequence, every other adaptor may comprise a stabilizing sequence, every third adaptor may comprise a stabilizing sequence, or every fourth, fifth, sixth, seventh, eighth, ninth or tenth adaptor may comprise a stabilizing sequence.

As described, the stabilizing sequences can comprise true palindromic sequences such as, e.g., GCTCGAGCTCGAGC (SEQ ID NO. 5), or the stabilizing sequences can comprise a palindromic sequence that is interrupted by a non-palindromic sequence of nucleotides (sequences that are complementary rather than true palindromes); for example, GCTCGAGTGTTGTCTCGAGC (SEQ ID NO. 6) (where the palindromic sequences are underlined). Thus, the stabilizing sequences may be true palindromes, or complementary sequences separated by a few to many non-complementary or non-palindromic sequences. As shown in library construct 360, the complementary sequences can be separated substantially by one, two or more adaptors without stabilizing sequences (306) and target nucleic acid sequences (301).

Alternatively or in addition, stabilizing sequences may comprise sequences or modified or unmodified nucleotides that are available for crosslinking. For example, alkylating agents such as 1,3-bis(2-chloroethyl)-1-nitrosurea and nitrogen mustard can cross link with DNA at the N7 position of guanine on opposite strands (see, e.g., U.S. Pat. No. 5,849,482). In another example, 5-bromo dU can be incorporated into the amplicon during circle-dependent replication, and will form intramolecular crosslinks within an amplicon upon exposure to ultraviolet light. In addition, cisplatin and its derivatives; psoralens in combination with ultraviolet wavelengths; and aldehydes such as acrolen and crotonaldehyde are known to be useful for crosslinking nucleic acids. As described herein, the monomers of the concatamers described herein can have 1, 2, 3, 4 or more stabilization sequences, depending on the number of adapters, the number of DNBs to be made, etc. In some cases, the same stabilization sequence can be used in each adapter, while alternate embodiments utilize different stabilization sequences. In one embodiment, further described herein, 4 adapters are utilized with 3 of the adapters containing the same palindromic sequence. As will be appreciated by those in the art, all and any combinations of these elements are possible.

In further embodiments, stabilizing sequences of the invention do not comprise palindromic sequences, but different adaptors comprise sequences that are complementary to one another, such that in a concatemer comprising such adaptors, those complementary sequences will hybridize to one another and thus direct the secondary structure of the concatemer.

In some embodiments, a single adaptor of a target nucleic acid template of the invention will comprise a stabilizing sequence and/or an anchor site. In some embodiments, multiple adaptors of a target nucleic acid template will comprise a stabilizing sequence and/or an anchor site. In some embodiments, fewer than all adaptors in a target nucleic acid template will comprise both a stabilizing sequence and an anchor site, and some adaptors will comprise only an anchor site. In some embodiments, adaptors will comprise one or more anchor sites and/or one or more stabilizing sequences. In further embodiments, adaptors can further comprise primer sites for reactions such as PCR and circle dependent replication (such as RCR) reactions. In certain embodiments, target nucleic acid templates of the invention will comprise one, two, three, four or more adaptors, and less than all of these adaptors will comprise stabilizing sequences. For example, in embodiments comprising four adaptors, only one, two or three of the adaptors may contain one or more stabilizing sequences. In further embodiments, the stabilizing sequences will comprise palindromes, and in still further embodiments, all adaptors comprising stabilizing sequences will comprise the same palindrome. In still further embodiments, template nucleic acids of the invention will comprise one or more adaptors, and at least one of those one or more adaptors will comprise a sequence according to at least one of SEQ ID NOs: 1-9. As will be appreciated, any combination of adaptors comprising anchor sites, primer sites and stabilizing sequences is encompassed by the present invention.

As will be described in further detail below, nucleic acid templates of the invention can be used to generate concatemers. These concatemers are generally composed of repeating monomers, where each monomer is a nucleic acid template of the invention. Thus, concatemers of the invention contain tens to hundreds of repeating units of target sequence interspersed with adaptors. In some embodiments, “multi-strand” amplicons or concatemers are generated from nucleic acid templates of the invention. By initiating a circle dependent replication reaction at two or more sites on a circular nucleic acid template simultaneously, an amplicon comprising multiple concatemeric strands can be produced. When such multi-strand amplicons comprise stabilizing sequences according to the present invention, the different strands of the amplicon interact with each other, generally through hybridization of palindromic or otherwise complementary sequences on the different strands. Such interactions produce a more compact multi-strand than would result from a similar amplicon that did not comprise such stabilizing sequences.

In some embodiments, the present invention provides libraries comprising target nucleic acid templates and concatemers generated from such templates for use in multiple high-throughput sequencing methodologies. Such libraries of nucleic acid templates and concatemers will in some embodiments comprise target nucleic acids that together encompass all or part of an entire genome. That is, by using a sufficient number of starting genomes (e.g. cells), combined with random fragmentation, the resulting target nucleic acids of a particular size that are used to create the circular templates of the invention sufficiently “cover” the genome, although as will be appreciated, on occasion, bias may be introduced inadvertently that prevent the entire genome from being represented. Some or all of this bias may in further embodiments be reduced or eliminated by utilizing the compositions and methods described herein. The libraries may contain in some exemplary embodiments from one to one million genome equivalents. In further exemplary embodiments, libraries of the invention comprise about 1 to about 1000, about 5 to about 500, about 10 to about 250, about 15 to about 200, about 20 to about 100, about 30 to about 75, and about 40 to about 50 genome equivalents. In certain exemplary embodiments, libraries of the invention comprise about five to about fifteen genome equivalents.

In one aspect, the present invention provides concatemers comprising both stabilizing sequences and anchor sites. In general, the stabilizing sequences and the anchor sites are contained in adaptors of the concatemer. In some embodiments, the secondary structure is directed at least in part by the stabilizing sequences of the adaptors such that the anchor sites and the primer sites for amplification reactions are free of steric hindrance from the secondary structure of the concatemer. By remaining free of steric hindrance, these anchor sites and primer sites are more accessible for binding to probes and enzymes respectively, thus increasing the efficiency of the respective sequencing and amplification reactions. In further embodiments, stabilizing sequences of different adaptors within a concatemer of the invention interact with each other to create a more compact and stable nucleic acid nanoball than is seen when such stabilizing sequences are not included in the concatemer. This favoring of intramolecular interactions within nucleic acid nanoballs of the invention can also serve to reduce intermolecular interactions between nanoballs, which can in some embodiments improve representation of nucleic acid nanoballs in the plurality and reduce bias in large-scale sequencing reactions. For example (and without being bound to a particular mechanism of action), in some instances nucleic acid nanoballs containing certain sequence elements, such as stretches of tandem repeats, may be more likely to interact with other nanoballs. Such intermolecular interactions would result in a lowered efficiency in sequencing and/or amplification reactions utilizing these nanoballs, because many of the binding sites for primers, sequencing probes, anchor probes, and enzymes would be inaccessible. In addition, since different nanoballs will often comprise different target sequences, interaction between different nanoballs could result in artifacts or inconsistencies in any sequence reads or amplification products that result from such nanoballs. Thus, stabilizing sequences and adaptors that favor intramolecular over intermolecular interactions can help improve stability and efficiency of sequencing and amplification reactions conducted according to the present invention over reactions on template nucleic acids and nanoballs that do not comprise such stabilizing sequences and adaptors.

In some embodiments, sequencing bias against repetitive elements can be reduced through the use of a library of constructs comprising adaptor sequences that have a demonstrated efficiency in a biochemical reaction (e.g., a polymerase reaction or a binding and ligation reaction). Bias against amplification and/or initiation of a sequencing reaction can result when sequence context impacts on the initiation or efficiency of such biochemical reactions. In sequencing complex target nucleic acids, such as a mammalian genome, high throughput technologies require that thousands of copies of millions of nucleic acid molecules must be created and available for interrogation as discrete entities, e.g., available at discrete spatial locations on a substrate. Bias due to sequence context can thus have serious ramifications on the completion of sequencing such a complex molecule. One example of the impact of sequence context is a reduced efficiency of primer and/or polymerase binding to a construct due to secondary or tertiary structures within the construct. Another example is intermolecular interactions between amplicons with complementary sequences can hinder access to specific sequences within the amplicon. Use of adaptors demonstrating reaction efficiency in multiple sequence contexts with repetitive elements such as homopolymers, Alu repeats, and the like can help to reduce sequence-specific bias in amplicons produced from such a library, thus decreasing overall bias in sequencing. Adaptors and stabilizing sequences of the invention described herein can help reduce bias that results from the presence of repetitive elements within the target nucleic acids from which the DNBs of the invention are produced.

In some embodiments, concatemers of the invention are disposed on the surface of a substrate. Methods for making such compositions (also referred to herein as “arrays”) are described in further detail below. In certain embodiments, arrays of the invention comprise concatemers that are randomly disposed on an unpatterned or patterned surface. In certain embodiments, arrays of the invention comprise concatemers that are disposed in known locations on an unpatterned or patterned surface. Arrays of the invention may comprise concatemers fixed to surface by a variety of techniques, including covalent attachment and non-covalent attachment. In one embodiment, a surface may include capture probes that form complexes, e.g., double stranded duplexes, with component of a polynucleotide molecule, such as an adaptor oligonucleotide. In other embodiments, capture probes may comprise oligonucleotide clamps, or like structures, that form triplexes with adaptors, as described in Gryaznov et al, U.S. Pat. No. 5,473,060, which is hereby incorporated in its entirety for all purposes and in particular for all teachings related to arrays. Arrays of use in the present invention are described in U.S. application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188 all of which are hereby incorporated by reference in their entirety, and particularly for all disclosures related to arrays of nucleic acid nanoballs according to the present invention.

### III. Making Compositions of the Invention

The present invention provides methods for producing compositions of the invention, including methods for producing circular nucleic acid templates, concatemers generated from circular nucleic acid templates, and arrays of concatemers disposed on the surface of a substrate.

In one aspect, the present invention provides methods for the construction of circular nucleic acid templates that are used in amplification reactions that utilize such circular templates to create concatamers of the monomeric circular templates, forming “DNA nanoballs”, described below, which find use in a variety of sequencing and genotyping applications. As discussed above, circular or linear constructs of the invention comprise target nucleic acid sequences, generally fragments of genomic DNA (although as described herein, other templates such as cDNA can be used), with interspersed exogenous nucleic acid adaptors. The present invention provides methods for producing nucleic acid template constructs in which each subsequent adaptor is added to a target sequence at a defined position and also optionally in a defined orientation in relation to one or more previously inserted adaptors. These nucleic acid template constructs are generally circular nucleic acids (although in certain embodiments the constructs can be linear) that include target nucleic acids with multiple interspersed adaptors. These adaptors, as described herein, are exogenous sequences used in the sequencing and genotyping applications, and usually contain a restriction endonuclease site, particularly for enzymes such as Type IIs enzymes that cut outside of their recognition site. For ease of analysis, the reactions of the invention generally utilize embodiments in which the adaptors are inserted in particular orientations, rather than randomly.

Methods for creating nucleic acid templates of the invention are described for example in U.S. application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, all of which are hereby incorporated by reference in their entirety, and particularly for all disclosure related to the construction of nucleic acid templates of the invention, including the insertion of multiple interspersed adaptors.

Nucleic acid templates of the invention are generally created from target nucleic acids. As discussed above, target nucleic acids are nucleic acids of interest. In certain aspects of the invention, target nucleic acids are genomic nucleic acids, generally double stranded DNA obtained from a plurality of cells. In some embodiments, such genomic DNA is obtained from about 10 to 100 to 1000 or more cells. The use of a plurality of cells provides a level of redundancy that allows for extensive sequencing coverage of the genome. The genomic nucleic acid can be fragmented into appropriate sizes for generating nucleic acid templates of the invention using standard techniques such as physical or enzymatic fractionation, which can be further combined with size fractionation methods. Such fragmentation methods are known in the art and described herein. In some embodiments, such target sequence fragments can be further processed to improve the efficiency of later reactions to insert one or more adaptors. For example, many techniques used to fragment (also referred to herein as “fractionate”) nucleic acids result in a combination of lengths and chemistries on the termini of the fragments. For example, the termini may contain overlaps, and for many purposes, blunt ends of the double stranded fragments are preferred. Producing such blunt ends can be accomplished using known techniques such as a polymerase and dNTPs. Similarly, the fractionation techniques may also result in a variety of termini, such as 3′ and 5′ hydroxyl groups and/or 3′ and 5′ phosphate groups. In some embodiments, it is desirable to enzymatically alter these termini. For example, to prevent the ligation of multiple fragments without the adaptors, it can be desirable to alter the chemistry of the termini such that the correct orientation of phosphate and hydroxyl groups is not present, thus preventing “polymerization” of the target sequences. The control over the chemistry of the termini can be provided using methods known in the art. For example, in some circumstances, the use of phosphatase eliminates all the phosphate groups, such that all ends contain hydroxyl groups. Each end can then be selectively altered to allow ligation between the desired components. Methods for producing and processing nucleic acid fragments are known in the art and are also described in U.S. application Ser. Nos. 12/265,593; 12/266,385; 12/329,365; and 12/335,188, each of which is hereby incorporated by reference in its entirety for all purposes and in particular for all teachings related to fragmenting nucleic acids, processing nucleic acid fragments, and constructing nucleic acid templates of the invention.

In one embodiment, after fragmenting, (and in fact before or after any step in the methods for constructing template nucleic acids described herein and in the incorporated references) an amplification step can be applied to the population of fragmented nucleic acids to ensure that a large enough concentration of all the fragments is available for subsequent steps of creating the decorated nucleic acids of the invention and using those nucleic acids for obtaining sequence information. Such amplification methods are well known in the art and include without limitation: polymerase chain reaction (PCR), ligation chain reaction (sometimes referred to as oligonucleotide ligase amplification OLA), cycling probe technology (CPT), strand displacement assay (SDA), transcription mediated amplification (TMA), nucleic acid sequence based amplification (NASBA), rolling circle amplification (RCA) (for circularized fragments), and invasive cleavage technology.

In general, nucleic acid templates of the invention are constructed by inserting adaptors into target sequences. In one exemplary embodiment, nucleic acid templates of the invention are created using a method in first and second adaptor arms of a first adaptor are ligated to the ends of a target nucleic acid to form a first linear construct. This first adaptor will in many embodiments comprise a restriction endonuclease recognition site. The first linear construct is circularized, and the resultant first circular construct is cut with a restriction endonuclease that binds to the restriction endonuclease recognition site in the first adaptor and cuts in the target nucleic acid, producing a second linear construct. The first and second adaptor arms of a second adaptor are then added to the termini of the second linear construct, and again, the second adaptor may comprise a restriction endonuclease recognition site. These steps can be repeated multiple times to insert the desired number of adaptors into the target nucleic acid.

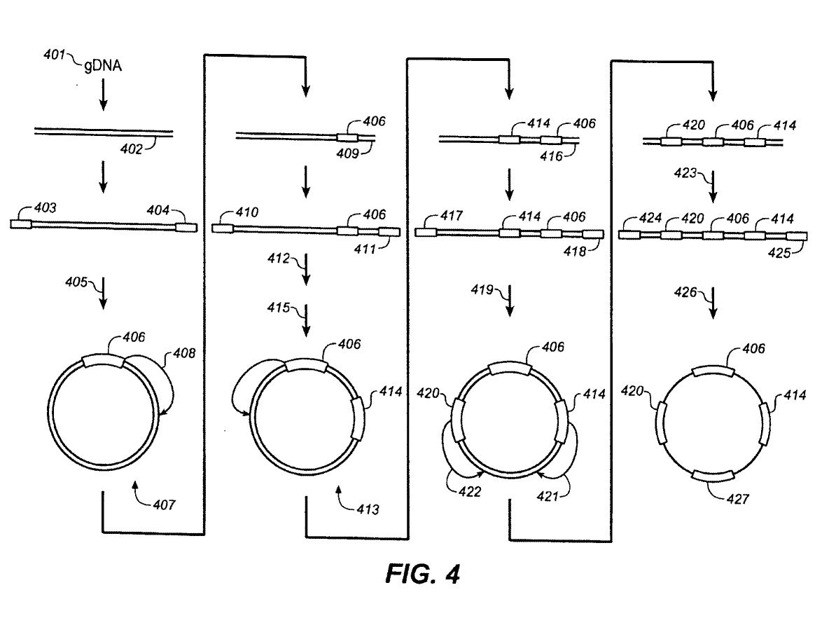

FIG. 4 is a schematic representation of one aspect of a method for assembling adaptor/target nucleic acid templates (also referred to herein as “target library constructs”, “library constructs” and all grammatical equivalents). DNA, such as genomic DNA 401, is isolated and fragmented into target nucleic acids 402 using standard techniques. The fragmented target nucleic acids 402 are then in some embodiments (as described herein) repaired so that the 5′ and 3′ ends of each strand are flush or blunt ended.

In the exemplary method illustrated in FIG. 4, a first (403) and second arm (404) of a first adaptor is ligated to each target nucleic acid, producing a target nucleic acid with adaptor arms ligated to each end.

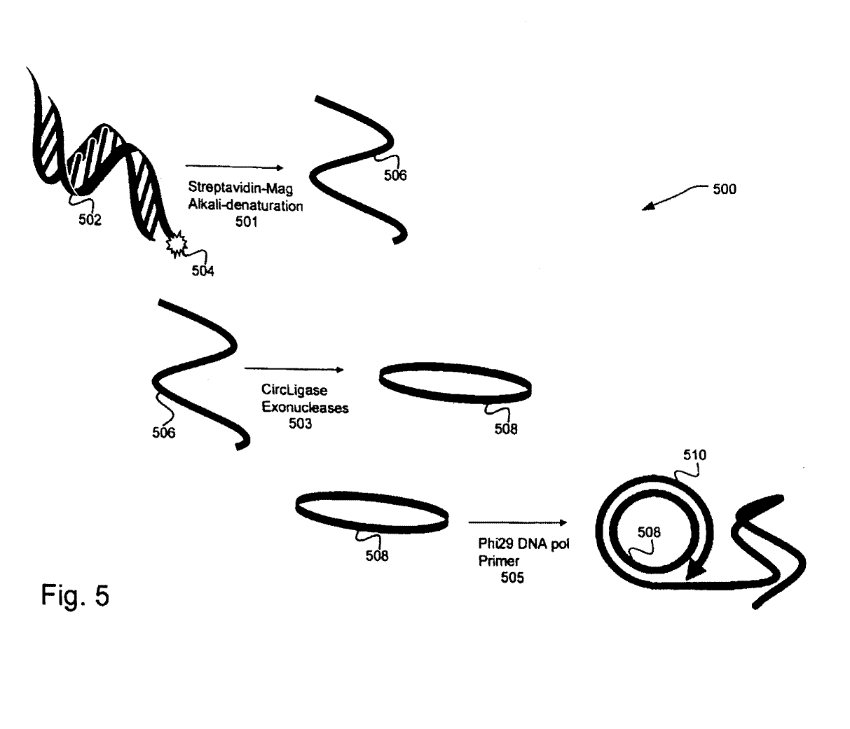

After creating a linear construct comprising a target nucleic acid and with an adaptor arm on each terminus, the linear target nucleic acid is circularized (405), a process that will be discussed in further detail herein, resulting in a circular construct 407 comprising target nucleic acid and an adaptor. Note that the circularization process results in bringing the first and second arms of the first adaptor together to form a contiguous first adaptor (406) in the circular construct. In some embodiments, the circular construct 407 is amplified, such as by circle dependent amplification, using, e.g., random hexamers and φ29 or helicase (an exemplary embodiment is illustrated in FIG. 5). Alternatively, target nucleic acid/adaptor structure may remain linear, and amplification may be accomplished by PCR primed from sites in the adaptor arms. The amplification preferably is a controlled amplification process and uses a high fidelity, proof-reading polymerase, resulting in a sequence-accurate library of amplified target nucleic acid/adaptor constructs where there is sufficient representation of the genome or one or more portions of the genome being queried.

Similar to the process for adding the first adaptor, as is further illustrated in FIG. 4, a second set of adaptor arms (410) and (411) can be added to each end of the linear molecule (409) and then ligated (412) to form the full adaptor (414) and circular molecule (413). Again, a third adaptor can be added to the other side of adaptor (409) by utilizing a Type IIs endonuclease that cleaves on the other side of adaptor (409) and then ligating a third set of adaptor arms (417) and (418) to each terminus of the linearized molecule. Finally, a fourth adaptor can be added by again cleaving the circular construct and adding a fourth set of adaptor arms to the linearized construct. The embodiment pictured in FIG. 4 is a method in which Type IIs endonucleases with recognition sites in adaptors (420) and (414) are applied to cleave the circular construct. The recognition sites in adaptors (420) and (414) may be identical or different. Similarly, the recognition sites in all of the adaptors illustrated in FIG. 4 may be identical or different.

Further embodiments and examples of methods of constructing nucleic acid templates of the invention are described in U.S. application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, each of which is herein incorporated by reference in its entirety for all purposes and in particular for all teachings related to constructing nucleic acid templates of the invention.

In further embodiments, after the desired number of adaptors are inserted into a target sequence, single stranded nucleic acid circles are formed from the constructs. In some exemplary embodiments, the final linear construct in FIG. 4 is a double stranded molecule. The strands of such a double stranded molecule can be separated to form single stranded constructs, and then those single stranded constructs are circularized using methods known in the art, including circularization through the use of a CircLigase enzyme. By “separating” the strands of a double stranded molecule as used herein is meant to encompass methods such as denaturing, separating strands by attaching a biotin molecule to one strand and utilizing streptavidin coated beads to separate the strand, and similar methods known in the art. In some exemplary embodiments, the final linear construct is circularized to form a double stranded circular molecule, and this double stranded molecule is then denatured to form single stranded circles.

Nucleic acid templates of the invention may be double stranded or single stranded, and they may be linear or circular. In some embodiments, libraries of nucleic acid templates are generated, and in further embodiments, the target sequences contained among the different templates in such libraries together cover all or part of an entire genome. As will be appreciated, these libraries of nucleic acid templates may comprise diploid genomes or they may be processed using methods known in the art to isolate sequences from one set of parental chromosomes over the other. As will also be appreciated by those of skill in the art, single stranded circular templates in libraries of the invention may together comprise both strands of a chromosome or chromosomal region (i.e., both “Watson” and “Crick” strands), or circles comprising sequences from one strand or the other may be isolated into their own libraries using methods known in the art.

IIIA. Methods for Adding Stabilizing Sequences to Compositions of the Invention

In some aspects, stabilizing sequences are incorporated into template nucleic acids of the invention. As described above, such stabilizing sequences may include palindromic sequences. Template nucleic acids comprising multiple stabilizing sequences may also include stabilizing sequences with complementary sequences, such that different stabilizing sequences are able to hybridize to each other.

In some embodiments, stabilizing sequences are designed into adaptors of the invention, such that the stabilizing sequences are incorporated into template nucleic acids upon insertion of those adaptors into the target sequences.

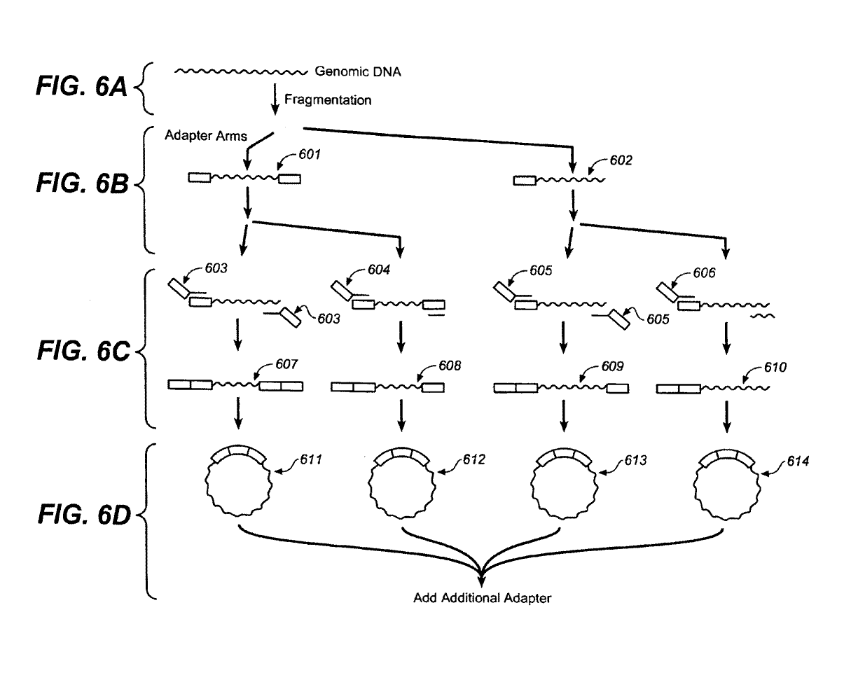

In some embodiments, stabilizing sequences are not originally part of adaptors inserted into target sequences, but are incorporated into (or adjacent to) the adaptors during the process of constructing the template nucleic acid construct. Exemplary embodiments of such a method are illustrated in FIG. 6. Genomic DNA (or other target sequences) are fragmented (if required) and then adaptors are ligated to the fragments. As depicted in FIG. 6, first and second adaptor arms (which together form a complete adaptor) can each be ligated to one end of the target sequence, or a complete adaptor can be added in a single ligation to one terminus of the fragment (note that the depiction herein on the “upstream” side of the target sequence is exemplary only). Once the adaptor or adaptor arms are added, an amplification reaction using primers plus “tails” comprising all or part of the stabilizing sequence can be conducted. As shown in line (c), this can be done with both primers comprising a “partial-tail” (see 603 and 605), e.g. each tail comprises part of the stabilizing sequence that together form the complete stabilizing sequence. Alternatively, only one of the primers may comprise a “full tail”, and this tail has the complete stabilizing sequence (see 604 and 606). After amplification, the resultant amplification products are circularized to form circular templates (see line (d)). These circular templates can be subjected to one or more cycles of the steps shown in lines (b) through (d) of FIG. 6 to insert the desired number of additional adaptors. While FIG. 6 depicts the situation where the addition of stabilizing sequence occurs during the addition of the “first” adaptor, as will be appreciated by those in the art, any or all of the embodiments pictured in FIG. 6 can be conducted with the addition of the second, third or fourth adaptor, or any combination thereof. Thus, the first adaptor may follow an “adaptor arm-two primers with half tails” technique (i.e., 603), and the addition of the second adaptor may not utilize the addition of tails (e.g. the adaptor may already comprise a stabilizing sequence in its sequence or the adaptor may not include a stabilizing sequence at all), and the third adaptor can follow a “adapter arm-one primer with full tail” technique (i.e, 604), etc. Thus all combinations are possible.

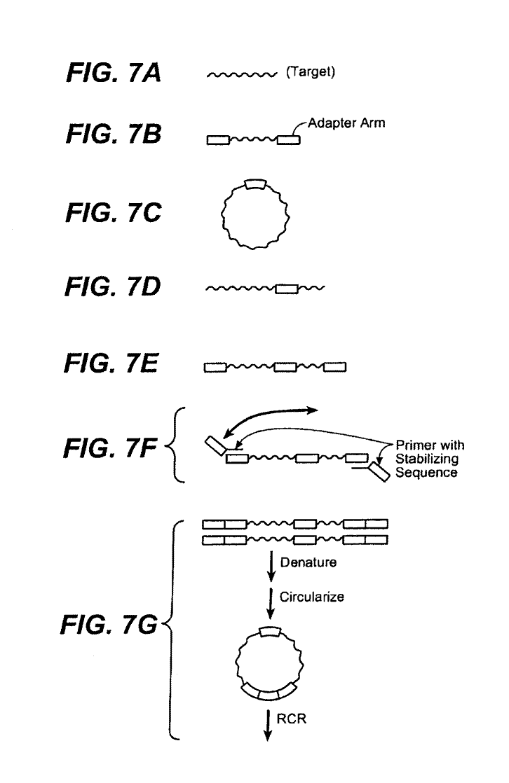

A further exemplary embodiment of incorporating stabilizing sequences into template nucleic acids of the invention is pictured in FIG. 7. In this embodiment, a target sequence is ligated to two arms of a first adaptor in step (b) using methods such as those described above. This first adaptor comprises a recognition site for a Type IIs restriction endonuclease. The resultant construct is then circularized in step (c) and then cleaved with the Type IIs restriction endonuclease to produce the construct in step (d). Two arms of a second adaptor are ligated to the linearized construct in step (d) to produce the construct in step (e). The construct in step (e) is then amplified in a template dependent nucleic acid amplification reaction such as PCR. The amplification is conducted using primers that include stabilizing sequences—as a result of the amplification, the stabilizing sequences are incorporated into the amplified product. This amplification product (pictured in (g)) can then be used to generate nucleic acid nanoballs of the invention. FIG. 7 depicts an exemplary embodiment in which the double stranded amplification products are denatured and then circularized, and the resultant single stranded circles are then subjected to a circle dependent replication method (such as RCR), to produce concatemers. As will be discussed in further detail below, concatemers can also be generated by circularizing the double stranded amplification product, nicking the double stranded circle, and then conducting a circle dependent replication method on the nicked circle.

As will be appreciated, although the embodiment pictured in FIG. 7 illustrates a target nucleic acid template with two adaptors, the present invention encompasses target nucleic acid templates with two, three, four or more adaptors. In addition, although the embodiment pictured in FIG. 7 incorporates the stabilizing sequences in the second adaptor, it will be appreciated that similar methods can be used to incorporate such sequences into any adaptor in a template nucleic acid, and that more than one of the adaptors in a template nucleic acid can have such sequences incorporated.

IIIB. Model Systems for Identifying Adaptors Useful in Reducing Sequencing and Amplification Bias

The selection of adaptors of the invention that can improve the efficiency of amplicon production and/or sequencing reactions can be performed using model systems of the invention that provide a way to assay and measure the efficiency of such processes. FIG. 5 illustrates four different adaptor recognition sequences that are designed to be located in an unimpeded binding region of the adaptor, and which are used in exemplary assays of the invention. The efficiency of amplicon production for each construct can be determined through direct hybridization of the differentially labeled probes or detection of the percentage of each of the amplicon populations. Efficiency of production can be determined using metrics such as the number of actual amplicons produced, fraction of amplicons comprising each adaptor, overall strength of probe signal for each set of amplicons, percentage of each nucleotide detected, and the like.

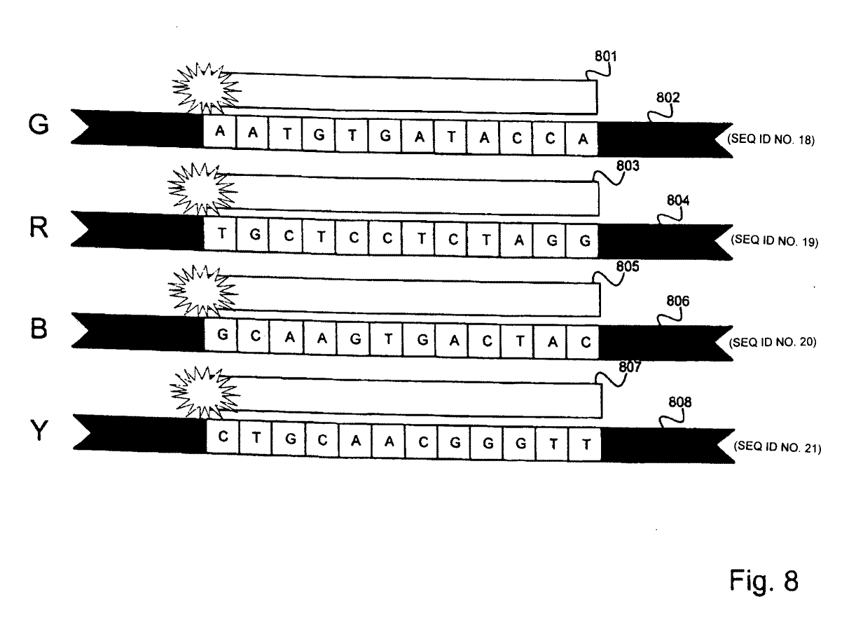

FIG. 8 is a schematic illustration of one model system used to assess amplicon quantity and quality when using engineered adaptors in either random or specific sequence contexts in sequencing constructs. The constructs are provided here in an initial concentration of 1:1:1:1, which should result in an approximately equal distribution of the number of amplicons produced if the efficiency of production is substantially the same for each construct. Model probes 801, 803, 805 and 807 are labeled G, R, B, or Y corresponding to green (Cy5), red (Texas red), blue (FITC) or yellow (Cy3). The structures numbered 802, 804, 806, and 808 correspond to portions of the binding regions four adaptors that have sequences engineered to bind to (are complementary to) one of the four model probes. The probe-binding engineered sequences illustrated in FIG. 8 are 12-mer sequences, but the length of this region can be varied to include either longer sequences, (e.g., 13-200 nucleotides) or shorter (e.g., 4-11 nucleotides), depending upon the desired region to be tested. This assay can confirm that a binding region within an adaptor is indeed unimpeded, which can be demonstrated by the efficiency of amplicon production and/or use in the model system.

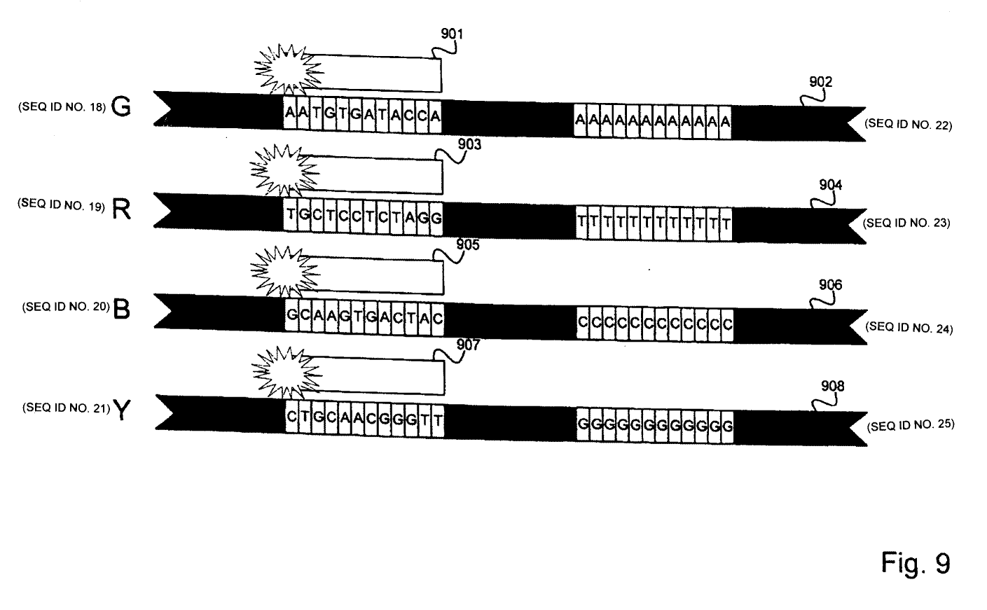

These model structures can be used in various combinations and sequence contexts to determine the quality and efficiency of adaptor sequences in amplicon production and/or use. This includes testing the effects of stabilizing sequences in adaptors to prevent intermolecular interactions between amplicons. In one example, the model probe sequences may correspond to four different adaptors used in a sequence specific context in an amplicon, e.g., to identify adaptors that will work particularly well in difficult sequencing regions such as tandem repeats. In another example, the model probe sequences can be used in four identical adaptors and different target sequences in each construct, to measure efficiency of a single adaptor with random sequences in amplicon production and/or use. In a specific example, illustrated in FIG. 9, the sequence context of the adaptor binding region is specifically engineered to determine the efficiency of one or more adaptors in a specific sequence context. In this example, the probe binding regions are placed upstream of poly-nucleotide repeats—in this specific example, a 12 nucleotide repeat placed at a pre-determined distance from the 3′ end of the probe hybridization sequence. This process allows for identification of adaptor sequences that are useful for sequencing specific areas of interest such as repetitive element regions of a genome, and for the ultimate design of amplicons that address sequencing bias and are produced efficiently.

Efficiency in amplicon production using a specific construct (e.g., by amplification or replication) can be assessed by direct measurement of the binding of a probe to each amplicon produced, and the signal produced by each amplicon population as measured by the probes. This assessment can be made on an individual basis for each amplicon population comprising a single adaptor, or multiple production and hybridization reactions can be carried out simultaneously, and the percentage of each population compared to determine the efficiency of each adaptor in the sequence context of each amplicon.

Efficiency in the sequencing reaction can be predicted by varying placement of the probe binding region used for the biochemical sequencing reaction, and measuring the hybridization of the probes to each amplicon under conditions similar to those that are used for the sequencing reaction. Again, measurement of hybridization can be performed on an individual basis for each amplicon population comprising a single adaptor, or multiple production and hybridization reactions can be carried out simultaneously, and the percentage of each population compared to determine the efficiency of each adaptor in the sequence context of each amplicon.

In addition to detecting quantity of amplicons produced, the assay of the invention can be used to assess the quality of the amplicons produced using an assessment of color purity. A single amplicon produced in this assay should have only one type of adaptor, and thus one engineered sequence for probe-binding. Once the amplicons are arrayed and the model probes are allowed to hybridize to the amplicons, the amplicons are imaged and percent color purity is assessed. Since each amplicon should only bind one model probe, the amplicon color image should be pure (that is, pure red, green, blue or yellow). On the other hand, impure color images result from, among other things, intermolecular interactions between amplicons, either prior to or after amplicon production.

The model system described is one method of assessing individual amplicon quality, and can be used as a model system to evaluate the effectiveness of stabilizing sequences (or other adaptor sequences) as well as be used for an initial quality step in an actual sequencing experiment. As a quality control measure, the model system can be used to identify amplicons that should not be read during the sequencing process. As will be appreciated, the variations of the exemplary model systems described herein, which are altered according to methods and principles known in the art, are encompassed by the present invention.

IIIC. Making Concatemers of the Invention

In one aspect, nucleic acid templates of the invention are used to generate nucleic acid nanoballs, which are also referred to herein as “DNA nanoballs,” “DNBs”, and “amplicons”. These nucleic acid nanoballs are generally concatemers comprising multiple copies of a nucleic acid template of the invention, although nucleic acid nanoballs of the invention may be formed from any nucleic acid molecule using the methods described herein.

In one aspect, rolling circle replication (RCR) is used to create concatemers of the invention. The RCR process has been shown to generate multiple continuous copies of the M13 genome. (Blanco, et al., (1989) J Biol Chem 264:8935-8940). In such a method, a nucleic acid is replicated by linear concatemerization. Guidance for selecting conditions and reagents for RCR reactions is available in many references available to those of ordinary skill, including U.S. Pat. Nos. 5,426,180; 5,854,033; 6,143,495; and 5,871,921, each of which is hereby incorporated by reference in its entirety for all purposes and in particular for all teachings related to generating concatemers using RCR or other methods.

Generally, RCR reaction components include single stranded DNA circles, one or more primers that anneal to DNA circles, a DNA polymerase having strand displacement activity to extend the 3′ ends of primers annealed to DNA circles, nucleoside triphosphates, and a conventional polymerase reaction buffer. Such components are combined under conditions that permit primers to anneal to DNA circle. Extension of these primers by the DNA polymerase forms concatemers of DNA circle complements. In some embodiments, nucleic acid templates of the invention are double stranded circles that are denatured to form single stranded circles that can be used in RCR reactions.

In some embodiments, amplification of circular nucleic acids may be implemented by successive ligation of short oligonucleotides, e.g., 6-mers, from a mixture containing all possible sequences, or if circles are synthetic, a limited mixture of these short oligonucleotides having selected sequences for circle replication, a process known as “circle dependent amplification” (CDA). “Circle dependant amplification” or “CDA” refers to multiple displacement amplification of a double-stranded circular template using primers annealing to both strands of the circular template to generate products representing both strands of the template, resulting in a cascade of multiple-hybridization, primer-extension and strand-displacement events. This leads to an exponential increase in the number of primer binding sites, with a consequent exponential increase in the amount of product generated over time. The primers used may be of a random sequence (e.g., random hexamers) or may have a specific sequence to select for amplification of a desired product. CDA results in a set of concatemeric double-stranded fragments being formed.

Concatemers may also be generated by ligation of target DNA in the presence of a bridging template DNA complementary to both beginning and end of the target molecule. A population of different target DNA may be converted in concatemers by a mixture of corresponding bridging templates.

In some aspects, concatemers are generated using two or more primer sequences. Each of the primers can function as a polymerization initiation site, resulting in the formation of a multi-strand amplicon. The use of primers of different sequence to initiate circle-dependent replication may decrease the likelihood that polymerization will be negatively biased due to sequence-specific interactions with the nucleotides within the template, and thus increase the potential for efficient amplicon production using a single template. Also, multi-strand amplicons may contain a greater number of copies of constituent sequences than single strand amplicons.

In some aspects, an amplicon may be created using a double-stranded circular template, which is then nicked at two or more sites. The nicked sites serve as polymerization initiation sites for circle-dependent replication, resulting in a multi-strand amplicon. Such nicking and polymerization can also decrease bias that may result due to inefficiency of polymerization initiation from a specific sequence within the circular template. Also, multi-strand amplicons may contain a greater number of copies of constituent sequences than single strand amplicons.

In some embodiments, a subset of a population of nucleic acid templates may be isolated based on a particular feature, such as a desired number or type of adaptor. This population can be isolated or otherwise processed (e.g., size selected) using conventional techniques, e.g., a conventional spin column, or the like, to form a population from which a population of concatemers can be created using techniques such as RCR.

Methods for forming DNBs of the invention are described in Published Patent Application Nos. WO2007120208, WO2006073504, WO2007133831, and US2007099208, and U.S. patent application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, all of which are incorporated herein by reference in their entirety for all purposes and in particular for all teachings related to forming DNBs.

IIID. Making Arrays of the Invention

In one aspect, DNBs of the invention are disposed on a surface to form a random array of single molecules. DNBs can be fixed to surface by a variety of techniques, including covalent attachment and non-covalent attachment. In one embodiment, a surface may include capture probes that form complexes, e.g., double stranded duplexes, with component of a polynucleotide molecule, such as an adaptor oligonucleotide. In other embodiments, capture probes may comprise oligonucleotide clamps, or like structures, that form triplexes with adaptors, as described in Gryaznov et al, U.S. Pat. No. 5,473,060, which is hereby incorporated in its entirety.

Methods for forming arrays of DNBs of the invention are described in Published Patent Application Nos. WO2007120208, WO2006073504, WO2007133831, and US2007099208, and U.S. patent application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, all of which are incorporated herein by reference in their entirety for all purposes and in particular for all teachings related to forming arrays of DNBs.

In some embodiments, a surface may have reactive functionalities that react with complementary functionalities on the polynucleotide molecules to form a covalent linkage, e.g., by way of the same techniques used to attach cDNAs to microarrays, e.g., Smirnov et al (2004), Genes, Chromosomes & Cancer, 40: 72-77; Beaucage (2001), Current Medicinal Chemistry, 8: 1213-1244, which are incorporated herein by reference. DNBs may also be efficiently attached to hydrophobic surfaces, such as a clean glass surface that has a low concentration of various reactive functionalities, such as —OH groups. Attachment through covalent bonds formed between the polynucleotide molecules and reactive functionalities on the surface is also referred to herein as “chemical attachment”.

In still further embodiments, polynucleotide molecules can adsorb to a surface. In such an embodiment, the polynucleotide molecules are immobilized through non-specific interactions with the surface, or through non-covalent interactions such as hydrogen bonding, van der Waals forces, and the like.

Attachment may also include wash steps of varying stringencies to remove incompletely attached single molecules or other reagents present from earlier preparation steps whose presence is undesirable or that are nonspecifically bound to surface.

In one aspect, DNBs on a surface are confined to an area of a discrete region. Discrete regions may be incorporated into a surface using methods known in the art and described further herein. In exemplary embodiments, discrete regions contain reactive functionalities or capture probes which can be used to immobilize the polynucleotide molecules.

The discrete regions may have defined locations in a regular array, which may correspond to a rectilinear pattern, hexagonal pattern, or the like. A regular array of such regions is advantageous for detection and data analysis of signals collected from the arrays during an analysis. Also, first- and/or second-stage amplicons confined to the restricted area of a discrete region provide a more concentrated or intense signal, particularly when fluorescent probes are used in analytical operations, thereby providing higher signal-to-noise values. In some embodiments, DNBs are randomly distributed on the discrete regions so that a given region is equally likely to receive any of the different single molecules. In other words, the resulting arrays are not spatially addressable immediately upon fabrication, but may be made so by carrying out an identification, sequencing and/or decoding operation. As such, the identities of the polynucleotide molecules of the invention disposed on a surface are discernable, but not initially known upon their disposition on the surface. In some embodiments, the area of discrete is selected, along with attachment chemistries, macromolecular structures employed, and the like, to correspond to the size of single molecules of the invention so that when single molecules are applied to surface substantially every region is occupied by no more than one single molecule. In some embodiments, DNBs are disposed on a surface comprising discrete regions in a patterned manner, such that specific DNBs (identified, in an exemplary embodiment, by tag adaptors or other labels) are disposed on specific discrete regions or groups of discrete regions.

In some embodiments, the area of discrete regions is less than 1 μm²; and in some embodiments, the area of discrete regions is in the range of from 0.04 μm² to 1 μm²; and in some embodiments, the area of discrete regions is in the range of from 0.2 μm² to 1 μm². In embodiments in which discrete regions are approximately circular or square in shape so that their sizes can be indicated by a single linear dimension, the size of such regions are in the range of from 125 nm to 250 nm, or in the range of from 200 nm to 500 nm. In some embodiments, center-to-center distances of nearest neighbors of discrete regions are in the range of from 0.25 μm to 20 μm; and in some embodiments, such distances are in the range of from 1 μm to 10 μm, or in the range from 50 to 1000 nm. Generally, discrete regions are designed such that a majority of the discrete regions on a surface are optically resolvable. In some embodiments, regions may be arranged on a surface in virtually any pattern in which regions have defined locations.

In further embodiments, molecules are directed to the discrete regions of a surface, because the areas between the discrete regions, referred to herein as “inter-regional areas,” are inert, in the sense that concatemers, or other macromolecular structures, do not bind to such regions. In some embodiments, such inter-regional areas may be treated with blocking agents, e.g., DNAs unrelated to concatemer DNA, other polymers, and the like.

A wide variety of supports may be used with the compositions and methods of the invention to form random arrays. In one aspect, supports are rigid solids that have a surface, preferably a substantially planar surface so that single molecules to be interrogated are in the same plane. The latter feature permits efficient signal collection by detection optics, for example. In another aspect, the support comprises beads, wherein the surface of the beads comprise reactive functionalities or capture probes that can be used to immobilize polynucleotide molecules.

In still another aspect, solid supports of the invention are nonporous, particularly when random arrays of single molecules are analyzed by hybridization reactions requiring small volumes. Suitable solid support materials include materials such as glass, polyacrylamide-coated glass, ceramics, silica, silicon, quartz, various plastics, and the like. In one aspect, the area of a planar surface may be in the range of from 0.5 to 4 cm². In one aspect, the solid support is glass or quartz, such as a microscope slide, having a surface that is uniformly silanized. This may be accomplished using conventional protocols, e.g., acid treatment followed by immersion in a solution of 3-glycidoxypropyl trimethoxysilane, N,N-diisopropylethylamine, and anhydrous xylene (8:1:24 v/v) at 80° C., which forms an epoxysilanized surface. e.g., Beattie et a (1995), Molecular Biotechnology, 4: 213. Such a surface is readily treated to permit end-attachment of capture oligonucleotides, e.g., by providing capture oligonucleotides with a 3′ or 5′ triethylene glycol phosphoryl spacer (see Beattie et al, cited above) prior to application to the surface. Further embodiments for functionalizing and further preparing surfaces for use in the present invention are described for example in U.S. patent application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, each of which is herein incorporated by reference in its entirety for all purposes and in particular for all teachings related to preparing surfaces for forming arrays and for all teachings related to forming arrays, particularly arrays of DNBs.

In embodiments of the invention in which patterns of discrete regions are required, photolithography, electron beam lithography, nano imprint lithography, and nano printing may be used to generate such patterns on a wide variety of surfaces, e.g., Pirrung et al, U.S. Pat. No. 5,143,854; Fodor et al, U.S. Pat. No. 5,774,305; Guo, (2004) Journal of Physics D: Applied Physics, 37: R123-141; which are incorporated herein by reference.

In one aspect, surfaces containing a plurality of discrete regions are fabricated by photolithography. A commercially available, optically flat, quartz substrate is spin coated with a 100-500 nm thick layer of photo-resist. The photo-resist is then baked on to the quartz substrate. An image of a reticle with a pattern of regions to be activated is projected onto the surface of the photo-resist, using a stepper. After exposure, the photo-resist is developed, removing the areas of the projected pattern which were exposed to the UV source. This is accomplished by plasma etching, a dry developing technique capable of producing very fine detail. The substrate is then baked to strengthen the remaining photo-resist. After baking, the quartz wafer is ready for functionalization. The wafer is then subjected to vapor-deposition of 3-aminopropyldimethylethoxysilane. The density of the amino functionalized monomer can be tightly controlled by varying the concentration of the monomer and the time of exposure of the substrate. Only areas of quartz exposed by the plasma etching process may react with and capture the monomer. The substrate is then baked again to cure the monolayer of amino-functionalized monomer to the exposed quartz. After baking, the remaining photo-resist may be removed using acetone. Because of the difference in attachment chemistry between the resist and silane, aminosilane-functionalized areas on the substrate may remain intact through the acetone rinse. These areas can be further functionalized by reacting them with p-phenylenediisothiocyanate in a solution of pyridine and N—N-dimethlyformamide. The substrate is then capable of reacting with amine-modified oligonucleotides. Alternatively, oligonucleotides can be prepared with a 5′-carboxy-modifier-c10 linker (Glen Research). This technique allows the oligonucleotide to be attached directly to the amine modified support, thereby avoiding additional functionalization steps.

In another aspect, surfaces containing a plurality of discrete regions are fabricated by nano-imprint lithography (NIL). For DNA array production, a quartz substrate is spin coated with a layer of resist, commonly called the transfer layer. A second type of resist is then applied over the transfer layer, commonly called the imprint layer. The master imprint tool then makes an impression on the imprint layer. The overall thickness of the imprint layer is then reduced by plasma etching until the low areas of the imprint reach the transfer layer. Because the transfer layer is harder to remove than the imprint layer, it remains largely untouched. The imprint and transfer layers are then hardened by heating. The substrate is then put into a plasma etcher until the low areas of the imprint reach the quartz. The substrate is then derivatized by vapor deposition as described above.

In another aspect, surfaces containing a plurality of discrete regions are fabricated by nano printing. This process uses photo, imprint, or e-beam lithography to create a master mold, which is a negative image of the features required on the print head. Print heads are usually made of a soft, flexible polymer such as polydimethylsiloxane (PDMS). This material, or layers of materials having different properties, are spin coated onto a quartz substrate. The mold is then used to emboss the features onto the top layer of resist material under controlled temperature and pressure conditions. The print head is then subjected to a plasma based etching process to improve the aspect ratio of the print head, and eliminate distortion of the print head due to relaxation over time of the embossed material. Random array substrates are manufactured using nano-printing by depositing a pattern of amine modified oligonucleotides onto a homogenously derivatized surface. These oligonucleotides would serve as capture probes for the RCR products. One potential advantage to nano-printing is the ability to print interleaved patterns of different capture probes onto the random array support. This would be accomplished by successive printing with multiple print heads, each head having a differing pattern, and all patterns fitting together to form the final structured support pattern. Such methods allow for some positional encoding of DNA elements within the random array. For example, control concatemers containing a specific sequence can be bound at regular intervals throughout a random array.

In still another aspect, a high density array of capture oligonucleotide spots of sub micron size is prepared using a printing head or imprint-master prepared from a bundle, or bundle of bundles, of about 10,000 to 100 million optical fibers with a core and cladding material. By pulling and fusing fibers a unique material is produced that has about 50-1000 nm cores separated by a similar or 2-5 fold smaller or larger size cladding material. By differential etching (dissolving) of cladding material a nano-printing head is obtained having a very large number of nano-sized posts. This printing head may be used for depositing oligonucleotides or other biological (proteins, oligopeptides, DNA, aptamers) or chemical compounds such as silane with various active groups. In one embodiment the glass fiber tool is used as a patterned support to deposit oligonucleotides or other biological or chemical compounds. In this case only posts created by etching may be contacted with material to be deposited. Also, a flat cut of the fused fiber bundle may be used to guide light through cores and allow light-induced chemistry to occur only at the tip surface of the cores, thus eliminating the need for etching. In both cases, the same support may then be used as a light guiding/collection device for imaging fluorescence labels used to tag oligonucleotides or other reactants. This device provides a large field of view with a large numerical aperture (potentially >1). Stamping or printing tools that perform active material or oligonucleotide deposition may be used to print 2 to 100 different oligonucleotides in an interleaved pattern. This process requires precise positioning of the print head to about 50-500 nm. This type of oligonucleotide array may be used for attaching 2 to 100 different DNA populations such as different source DNA. They also may be used for parallel reading from sub-light resolution spots by using DNA specific anchors or tags. Information can be accessed by DNA specific tags, e.g., 16 specific anchors for 16 DNAs and read 2 bases by a combination of 5-6 colors and using 16 ligation cycles or one ligation cycle and 16 decoding cycles. This way of making arrays is efficient if limited information (e.g., a small number of cycles) is required per fragment, thus providing more information per cycle or more cycles per surface.

In one aspect, multiple arrays of the invention may be placed on a single surface. For example, patterned array substrates may be produced to match the standard 96 or 384 well plate format. A production format can be an 8×12 pattern of 6 mm×6 mm arrays at 9 mm pitch or 16×24 of 3.33 mm×3.33 mm array at 4.5 mm pitch, on a single piece of glass or plastic and other optically compatible material. In one example each 6 mm×6 mm array consists of 36 million 250-500 nm square regions at 1 micrometer pitch. Hydrophobic or other surface or physical barriers may be used to prevent mixing different reactions between unit arrays.

Other methods of forming arrays of molecules are known in the art and are applicable to forming arrays of DNBs.

As will be appreciated, a wide range of densities of DNBs and/or nucleic acid templates of the invention can be placed on a surface comprising discrete regions to form an array. In some embodiments, each discrete region may comprise from about 1 to about 1000 molecules. In further embodiments, each discrete region may comprise from about 10 to about 900, about 20 to about 800, about 30 to about 700, about 40 to about 600, about 50 to about 500, about 60 to about 400, about 70 to about 300, about 80 to about 200, and about 90 to about 100 molecules.

In some embodiments, arrays of nucleic acid templates and/or DNBs are provided in densities of at least 0.5, 1, 2, 3, 4, 5, 6, 7, 8, 9, or 10 million molecules per square millimeter.

### IV. Using Compositions of the Invention

DNBs made according to the methods described herein offer an advantage in identifying sequences in target nucleic acids, because the adaptors contained in the DNBs provide points of known sequence that allow spatial orientation and sequence determination when combined with methods utilizing anchor and sequencing probes. In addition, DNBs described herein generally have conformations directed at least in part by sequences contained in their adaptors, and these conformations are such that sequencing bias is reduced, because binding sites for primers involved in sequencing reactions described herein are relatively free of steric hindrance by the secondary structure of the DNBs.

Methods of using DNBs in accordance with the present invention include sequencing and detecting specific sequences in target nucleic acids (e.g., detecting particular target sequences (e.g. specific genes) and/or identifying and/or detecting SNPs). The methods described herein can also be used to detect nucleic acid rearrangements and copy number variation. Nucleic acid quantification, such as digital gene expression (i.e., analysis of an entire transcriptome—all mRNA present in a sample) and detection of the number of specific sequences or groups of sequences in a sample, can also be accomplished using the methods described herein. Methods of using DNBs in sequencing reactions and in the detection of particular target sequences are also described in U.S. patent application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, each of which is herein incorporated by reference in its entirety for all purposes and in particular for all teachings related conducting sequencing reactions on DNBs of the invention. As will be appreciated, any of the sequencing methods described herein and known in the art can be applied to nucleic acid templates and/or DNBs of the invention in solution or to nucleic acid templates and/or DNBs disposed on a surface and/or in an array.

In one aspect, the present invention provides methods for identifying sequences of DNBs by utilizing sequencing by ligation methods. In one aspect, the present invention provides methods for identifying sequences of DNBs that utilize a combinatorial probe anchor ligation (cPAL) method. Generally, cPAL involves identifying a nucleotide at a detection position in a target nucleic acid by detecting a probe ligation product formed by ligation of at least one anchor probe and at least one sequencing probe. Such methods are described in U.S. patent application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, each of which is herein incorporated by reference in its entirety for all purposes and in particular for all teachings related to cPAL sequencing methods. Methods of the invention can be used to sequence a portion or the entire sequence of the target nucleic acid contained in a DNB, and many DNBs that represent a portion or all of a genome.

As discussed further herein, every DNB comprises repeating monomeric units, each monomeric unit comprising one or more adaptors and a target nucleic acid. The target nucleic acid comprises a plurality of detection positions. The term “detection position” refers to a position in a target sequence for which sequence information is desired. As will be appreciated by those in the art, generally a target sequence has multiple detection positions for which sequence information is required, for example in the sequencing of complete genomes as described herein. In some cases, for example in SNP analysis, it may be desirable to just read a single SNP in a particular area.

The present invention provides methods of sequencing by ligation that utilize a combination of anchor probes and sequencing probes. By “sequencing probe” as used herein is meant an oligonucleotide that is designed to provide the identity of a nucleotide at a particular detection position of a target nucleic acid. Sequencing probes hybridize to domains within target sequences, e.g. a first sequencing probe may hybridize to a first target domain, and a second sequencing probe may hybridize to a second target domain. The terms “first target domain” and “second target domain” or grammatical equivalents herein means two portions of a target sequence within a nucleic acid which is under examination. The first target domain may be directly adjacent to the second target domain, or the first and second target domains may be separated by an intervening sequence, for example an adaptor. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain. Sequencing probes can overlap, e.g. a first sequencing probe can hybridize to the first 6 bases adjacent to one terminus of an adaptor, and a second sequencing probe can hybridize to the 3rd-9th bases from the terminus of the adaptor (for example when an anchor probe has three degenerate bases). Alternatively, a first sequencing probe can hybridize to the 6 bases adjacent to the “upstream” terminus of an adaptor and a second sequencing probe can hybridize to the 6 bases adjacent to the “downstream” terminus of an adaptor.

Sequencing probes will generally comprise a number of degenerate bases and a specific nucleotide at a specific location within the probe to query the detection position (also referred to herein as an “interrogation position”).

In general, pools of sequencing probes are used when degenerate bases are used. That is, a probe having the sequence “NNNANN” is actually a set of probes of having all possible combinations of the four nucleotide bases at five positions (i.e., 1024 sequences) with an adenosine at the 6th position. (As noted herein, this terminology is also applicable to adaptor probes: for example, when an adaptor probe has “three degenerate bases”, for example, it is actually a set of adaptor probes comprising the sequence corresponding to the anchor site, and all possible combinations at 3 positions, so it is a pool of 64 probes).

In some embodiments, for each interrogation position, four differently labeled pools can be combined in a single pool and used in a sequencing step. Thus, in any particular sequencing step, 4 pools are used, each with a different specific base at the interrogation position and with a different label corresponding to the base at the interrogation position. That is, sequencing probes are also generally labeled such that a particular nucleotide at a particular interrogation position is associated with a label that is different from the labels of sequencing probes with a different nucleotide at the same interrogation position. For example, four pools can be used: NNNANN-dye1, NNNTNN-dye2, NNNCNN-dye3 and NNNGNN-dye4 in a single step, as long as the dyes are optically resolvable. In some embodiments, for example for SNP detection, it may only be necessary to include two pools, as the SNP call will be either a C or an A, etc. Similarly, some SNPs have three possibilities. Alternatively, in some embodiments, if the reactions are done sequentially rather than simultaneously, the same dye can be done, just in different steps: e.g. the NNNANN-dye1 probe can be used alone in a reaction, and either a signal is detected or not, and the probes washed away; then a second pool, NNNTNN-dye1 can be introduced.

In any of the sequencing methods described herein, sequencing probes may have a wide range of lengths, including about 3 to about 25 bases. In further embodiments, sequencing probes may have lengths in the range of about 5 to about 20, about 6 to about 18, about 7 to about 16, about 8 to about 14, about 9 to about 12, and about 10 to about 11 bases.

Sequencing probes of the present invention are designed to be complementary, and in general, perfectly complementary, to a sequence of the target sequence such that hybridization of a portion target sequence and probes of the present invention occurs. In particular, it is important that the interrogation position base and the detection position base be perfectly complementary and that the methods of the invention do not result in signals unless this is true.

In many embodiments, sequencing probes are perfectly complementary to the target sequence to which they hybridize; that is, the experiments are run under conditions that favor the formation of perfect basepairing, as is known in the art. As will be appreciated by those in the art, a sequencing probe that is perfectly complementary to a first domain of the target sequence could be only substantially complementary to a second domain of the same target sequence; that is, the present invention relies in many cases on the use of sets of probes, for example, sets of hexamers, that will be perfectly complementary to some target sequences and not to others.

In some embodiments, depending on the application, the complementarity between the sequencing probe and the target need not be perfect; there may be any number of base pair mismatches, which will interfere with hybridization between the target sequence and the single stranded nucleic acids of the present invention. However, if the number of mismatches is so great that no hybridization can occur under even the least stringent of hybridization conditions, the sequence is not a complementary target sequence. Thus, by “substantially complementary” herein is meant that the sequencing probes are sufficiently complementary to the target sequences to hybridize under normal reaction conditions. However, for most applications, the conditions are set to favor probe hybridization only if perfectly complementarity exists. Alternatively, sufficient complementarity is required to allow the ligase reaction to occur; that is, there may be mismatches in some part of the sequence but the interrogation position base should allow ligation only if perfect complementarity at that position occurs.

In some cases, in addition to or instead of using degenerate bases in probes of the invention, universal bases which hybridize to more than one base can be used. For example, inosine can be used. Any combination of these systems and probe components can be utilized.

Sequencing probes of use in methods of the present invention are usually detectably labeled. By “label” or “labeled” herein is meant that a compound has at least one element, isotope or chemical compound attached to enable the detection of the compound. In general, labels of use in the invention include without limitation isotopic labels, which may be radioactive or heavy isotopes, magnetic labels, electrical labels, thermal labels, colored and luminescent dyes, enzymes and magnetic particles as well. Dyes of use in the invention may be chromophores, phosphors or fluorescent dyes, which due to their strong signals provide a good signal-to-noise ratio for decoding. Sequencing probes may also be labeled with quantum dots, fluorescent nanobeads or other constructs that comprise more than one molecule of the same fluorophore. Labels comprising multiple molecules of the same fluorophore will generally provide a stronger signal and will be less sensitive to quenching than labels comprising a single molecule of a fluorophore. It will be understood that any discussion herein of a label comprising a fluorophore will apply to labels comprising single and multiple fluorophore molecules.

Many embodiments of the invention include the use of fluorescent labels. Suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue™, Texas Red, and others described in the 6th Edition of the Molecular Probes Handbook by Richard P. Haugland, hereby expressly incorporated by reference in its entirety for all purposes and in particular for its teachings regarding labels of use in accordance with the present invention. Commercially available fluorescent dyes for use with any nucleotide for incorporation into nucleic acids include, but are not limited to: Cy3, Cy5, (Amersham Biosciences, Piscataway, N.J., USA), fluorescein, tetramethylrhodamine-, Texas Red®, Cascade Blue®, BODIPY® FL-14, BODIPY®R, BODIPY® TR-14, Rhodamine Green™, Oregon Green® 488, BODIPY® 630/650, BODIPY® 650/665-, Alexa Fluor® 488, Alexa Fluor® 532, Alexa Fluor® 568, Alexa Fluor® 594, Alexa Fluor® 546 (Molecular Probes, Inc. Eugene, Oreg., USA), Quasar 570, Quasar 670, Cal Red 610 (BioSearch Technologies, Novato, Calif.). Other fluorophores available for post-synthetic attachment include, inter alia, Alexa Fluor® 350, Alexa Fluor® 532, Alexa Fluor® 546, Alexa Fluor® 568, Alexa Fluor® 594, Alexa Fluor® 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3.5, Cy5.5, and Cy7 (Amersham Biosciences, Piscataway, N.J. USA, and others). In some embodiments, the labels used include fluoroscein, Cy3, Texas Red, Cy5, Quasar 570, Quasar 670 and Cal Red 610 are used in methods of the present invention.

Labels can be attached to nucleic acids to form the labeled sequencing probes of the present invention using methods known in the art, and to a variety of locations of the nucleosides. For example, attachment can be at either or both termini of the nucleic acid, or at an internal position, or both. For example, attachment of the label may be done on a ribose of the ribose-phosphate backbone at the 2′ or 3′ position (the latter for use with terminal labeling), in one embodiment through an amide or amine linkage. Attachment may also be made via a phosphate of the ribose-phosphate backbone, or to the base of a nucleotide. Labels can be attached to one or both ends of a probe or to any one of the nucleotides along the length of a probe.

Sequencing probes are structured differently depending on the interrogation position desired. For example, in the case of sequencing probes labeled with fluorophores, a single position within each sequencing probe will be correlated with the identity of the fluorophore with which it is labeled. Generally, the fluorophore molecule will be attached to the end of the sequencing probe that is opposite to the end targeted for ligation to the anchor probe.

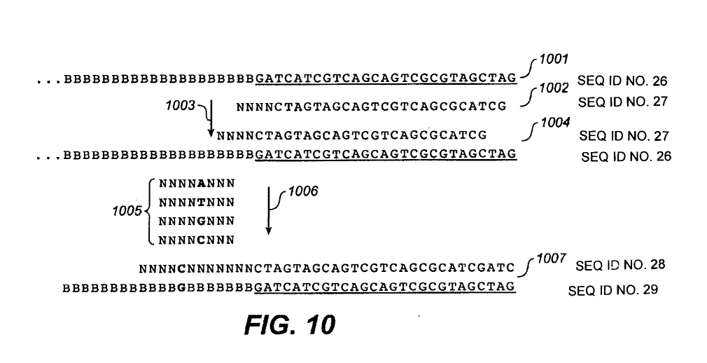

By “anchor probe” as used herein is meant an oligonucleotide designed to be complementary to at least a portion of an adaptor, referred to herein as “an anchor site”. Adaptors can contain multiple anchor sites for hybridization with multiple anchor probes, as described herein. As discussed further herein, anchor probes of use in the present invention can be designed to hybridize to an adaptor such that at least one end of the anchor probe is flush with one terminus of the adaptor (either “upstream” or “downstream”, or both). In further embodiments, anchor probes can be designed to hybridize to at least a portion of an adaptor (a first adaptor site) and also at least one nucleotide of the target nucleic acid adjacent to the adaptor (“overhangs”). As illustrated in FIG. 10, anchor probe 1002 comprises a sequence complementary to a portion of the adaptor. Anchor probe 1002 also comprises four degenerate bases at one terminus. This degeneracy allows for a portion of the anchor probe population to fully or partially match the sequence of the target nucleic acid adjacent to the adaptor and allows the anchor probe to hybridize to the adaptor and reach into the target nucleic acid adjacent to the adaptor regardless of the identity of the nucleotides of the target nucleic acid adjacent to the adaptor. This shift of the terminal base of the anchor probe into the target nucleic acid shifts the position of the base to be called closer to the ligation point, thus allowing the fidelity of the ligase to be maintained. In general, ligases ligate probes with higher efficiency if the probes are perfectly complementary to the regions of the target nucleic acid to which they are hybridized, but the fidelity of ligases decreases with distance away from the ligation point. Thus, in order to minimize and/or prevent errors due to incorrect pairing between a sequencing probe and the target nucleic acid, it can be useful to maintain the distance between the nucleotide to be detected and the ligation point of the sequencing and anchor probes. By designing the anchor probe to reach into the target nucleic acid, the fidelity of the ligase is maintained while still allowing a greater number of nucleotides adjacent to each adaptor to be identified. Although the embodiment illustrated in FIG. 10 is one in which the sequencing probe hybridizes to a region of the target nucleic acid on one side of the adaptor, it will be appreciated that embodiments in which the sequencing probe hybridizes on the other side of the adaptor are also encompassed by the invention. In FIG. 10, “N” represents a degenerate base and “B” represents nucleotides of undetermined sequence. As will be appreciated, in some embodiments, rather than degenerate bases, universal bases may be used. It will appreciated that FIG. 10 illustrates only one exemplary embodiment of sequencing by ligation methods of use in the present invention. Further embodiments are described in U.S. application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188, each of which is hereby incorporated in its entirety for all purposes and in particular for all teachings related to different embodiments of sequencing by ligation using combinations of anchor and sequencing probes.

Anchor probes of the invention may comprise any sequence that allows the anchor probe to hybridize to a DNB, generally to an adaptor of a DNB. Such anchor probes may comprise a sequence such that when the anchor probe is hybridized to an adaptor, the entire length of the anchor probe is contained within the adaptor. In some embodiments, anchor probes may comprise a sequence that is complementary to at least a portion of an adaptor and also comprise degenerate bases that are able to hybridize to target nucleic acid regions adjacent to the adaptor. In some exemplary embodiments, anchor probes are hexamers that comprise 3 bases that are complementary to an adaptor and 3 degenerate bases. In some exemplary embodiments, anchor probes are 8-mers that comprise 3 bases that are complementary to an adaptor and 5 degenerate bases. In further exemplary embodiments, particularly when multiple anchor probes are used, a first anchor probe comprises a number of bases complementary to an adaptor at one end and degenerate bases at another end, whereas a second anchor probe comprises all degenerate bases and is designed to ligate to the end of the first anchor probe that comprises degenerate bases. It will be appreciated that these are exemplary embodiments, and that a wide range of combinations of known and degenerate bases can be used to produce anchor probes of use in accordance with the present invention.

The present invention provides sequencing by ligation methods for identifying sequences of DNBs. In certain aspects, the sequencing by ligation methods of the invention include providing different combinations of anchor probes and sequencing probes, which, when hybridized to adjacent regions on a DNB, can be ligated to form probe ligation products. The probe ligation products are then detected, which provides the identity of one or more nucleotides in the target nucleic acid. By “ligation” as used herein is meant any method of joining two or more nucleotides to each other. Ligation can include chemical as well as enzymatic ligation. In general, the sequencing by ligation methods discussed herein utilize enzymatic ligation by ligases. Such ligases invention can be the same or different than ligases discussed above for creation of the nucleic acid templates. Such ligases include without limitation DNA ligase I, DNA ligase II, DNA ligase III, DNA ligase IV, E. coli DNA ligase, T4 DNA ligase, T4 RNA ligase 1, T4 RNA ligase 2, T7 ligase, T3 DNA ligase, and thermostable ligases (including without limitation Taq ligase) and the like. As discussed above, sequencing by ligation methods often rely on the fidelity of ligases to only join probes that are perfectly complementary to the nucleic acid to which they are hybridized. This fidelity will decrease with increasing distance between a base at a particular position in a probe and the ligation point between the two probes. As such, conventional sequencing by ligation methods can be limited in the number of bases that can be identified. The present invention increases the number of bases that can be identified by using multiple probe pools, as is described further herein.

A variety of hybridization conditions may be used in the sequencing by ligation methods of sequencing as well as other methods of sequencing described herein. These conditions include high, moderate and low stringency conditions; see for example Maniatis et al., Molecular Cloning: A Laboratory Manual, 2d Edition, 1989, and Short Protocols in Molecular Biology, ed. Ausubel, et al, which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays,” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions can be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

In a further aspect, sequences of DNBs are identified using sequencing methods other than sequencing by ligation. Such methods are known in the art and include, but are not limited to, hybridization-based methods, such as disclosed in Drmanac, U.S. Pat. Nos. 6,864,052; 6,309,824; and 6,401,267; and Drmanac et al, U.S. patent publication 2005/0191656, and sequencing by synthesis methods, e.g. Nyren et al, U.S. Pat. No. 6,210,891; Ronaghi, U.S. Pat. No. 6,828,100; Ronaghi et al (1998), Science, 281: 363-365; Balasubramanian, U.S. Pat. No. 6,833,246; Quake, U.S. Pat. No. 6,911,345; Li et al, Proc. Natl. Acad. Sci., 100: 414-419 (2003); Smith et al, PCT publication WO 2006/074351; and ligation-based methods, e.g. Shendure et al (2005), Science, 309: 1728-1739, Macevicz, U.S. Pat. No. 6,306,597, wherein each of these references is herein incorporated by reference in its entirety for all purposes and in particular teachings regarding the figures, legends and accompanying text describing the compositions, methods of using the compositions and methods of making the compositions, particularly with respect to sequencing.

In some embodiments, nucleic acid templates of the invention, as well as DNBs generated from those templates, are used in sequencing by synthesis methods. The efficiency of sequencing by synthesis methods utilizing nucleic acid templates of the invention is increased over conventional sequencing by synthesis methods utilizing nucleic acids that do not comprise multiple interspersed adaptors. Rather than a single long read, nucleic acid templates of the invention allow for multiple short reads that each start at one of the adaptors in the template. Such short reads consume fewer labeled dNTPs, thus saving on the cost of reagents. In addition, sequencing by synthesis reactions can be performed on DNB arrays, which provide a high density of sequencing targets as well as multiple copies of monomeric units. Such arrays provide detectable signals at the single molecule level while at the same time providing an increased amount of sequence information, because most or all of the DNB monomeric units will be extended without losing sequencing phase. The high density of the arrays also reduces reagent costs—in some embodiments the reduction in reagent costs can be from about 30 to about 40% over conventional sequencing by synthesis methods. In some embodiments, the interspersed adaptors of the nucleic acid templates of the invention provide a way to combine about two to about ten standard reads if inserted at distances of from about 30 to about 100 bases apart from one another. In such embodiments, the newly synthesized strands will not need to be stripped off for further sequencing cycles, thus allowing the use of a single DNB array through about 100 to about 400 sequencing by synthesis cycles.

Although much of the description of sequencing methods is provided in terms of nucleic acid templates of the invention, it will be appreciated that these sequencing methods also encompass identifying sequences in DNBs generated from such nucleic acid templates, as described herein.

For any of sequencing methods known in the art and described herein using nucleic acid templates of the invention, the present invention provides methods for determining at least about 10 to about 200 bases in target nucleic acids. In further embodiments, the present invention provides methods for determining at least about 20 to about 180, about 30 to about 160, about 40 to about 140, about 50 to about 120, about 60 to about 100, and about 70 to about 80 bases in target nucleic acids. In still further embodiments, sequencing methods are used to identify 5, 10, 15, 20, 25, 30 or more bases adjacent to one or both ends of each adaptor in a nucleic acid template of the invention.

### EXAMPLES

### Example 1

### Producing DNBs and Assessing DNB Quality

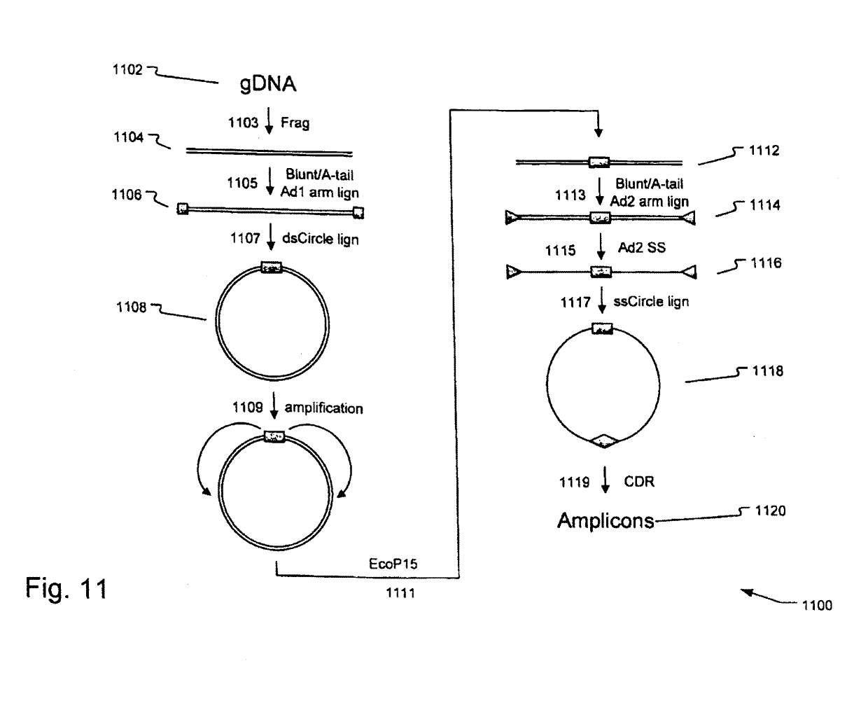

The following protocols are exemplary protocols for amplicon production, starting with a library construct such as that shown in FIG. 11 at 1106. The single-stranded linear library constructs are first subjected to amplification with a phosphorylated 5′ primer comprising a stabilizing sequence and a biotinylated 3′ primer, resulting in a library construct such as that shown at 502 in FIG. 5, where the biotin is shown at 504. Alternatively, the stabilizing sequences may be contained within one or more adaptors in the library construct. Methods for creating such library constructs are taught in U.S. application Ser. Nos. 11/679,124; 11/981,761; 11/981,661; 11/981,605; 11/981,793; 11/981,804; 11/451,691; 11/981,607; 11/981,767; 11/982,467; 11/451,692; 12/335,168; 11/541,225; 11/927,356; 11/927,388; 11/938,096; 11/938,106; 10/547,214; 11/981,730; 11/981,685; 11/981,797; 12/252,280; 11/934,695; 11/934,697; 11/934,703; 12/265,593; 12/266,385; 11/938,213; 11/938,221; 12/325,922; 12/329,365; and 12/335,188 and international application number PCT/US07/835,540; filed Nov. 2, 2007, all of which are incorporated by reference in their entirety for all purposes and in particular for all teachings related to creating library constructs.

Strand separation and purification of single-stranded library constructs: First, streptavidin magnetic beads were prepared by resuspending MagPrep-Streptavidin beads (Novagen Part. No. 70716-3) in 1× bead binding buffer (150 mM NaCl and 20 mM Tris, pH 7.5 in nuclease free water) in nuclease-free microfuge tubes. The tubes were placed in a magnetic tube rack, the magnetic particles were allowed to clear, and the supernatant was removed and discarded. The beads were then washed twice in 800 μl 1× bead binding buffer, and resuspended in 80 μl 1× bead binding buffer. Amplified library constructs from the PCR reaction were brought up to 60 μl volume, and 20 μl 4× bead binding buffer was added to the tube. The amplified library constructs were then added to the tubes containing the MagPrep beads, mixed gently, incubated at room temperature for 10 minutes and the MagPrep beads were allowed to clear. The supernatant was removed and discarded. The MagPrep beads (mixed with the amplified library constructs) were then washed twice in 800 μl 1× bead binding buffer. After washing, the MagPrep beads were resuspended in 80 μl 0.1 N NaOH, mixed gently, incubated at room temperature and allowed to clear. The supernatant was removed and added to a fresh nuclease-free tube. 4 μl 3M sodium acetate (pH 5.2) was added to each supernatant and mixed gently.

Next, 420 μl of PBI buffer (supplied with QIAprep PCR Purification Kits) was added to each tube, the samples were mixed and then were applied to QIAprep Miniprep columns (Qiagen Part No. 28106) in 2 ml collection tubes and centrifuged for 1 minutes at 14,000 rpm. The flow through was discarded, and 0.75 ml PE buffer (supplied with QIAprep PCR Purification Kits) was added to each column, and the column was centrifuged for an additional 1 minute. Again the flow through was discarded. The column was transferred to a fresh tube and 50 μl of EB buffer (supplied with QIAprep PCR Purification Kits) was added. The columns were spun at 14,000 for 1 minute to elute the single-stranded library constructs. The quantity of each sample was then measured.

Circularization of single-stranded template using a Single-stranded DNA Ligase: First, 10 pmole of the single-stranded linear library constructs was transferred to a nuclease-free PCR tube. Nuclease free water was added to bring the reaction volume to 30 μl, and the samples were kept on ice. Next, 4 μl 10× CircLigase Reaction Buffer (Epicentre Part. No. CL4155K), 2 μl 1 mM ATP, 2 μl 50 mM MnCl₂, and 2 μl single-stranded DNA ligase (CircLigase, 100 U/μl) (collectively, 4× Ligase Mix) were added to each tube, and the samples were incubated at 60° C. for 5 minutes. Another 10 μl of 4× Ligase Mix was added was added to each tube and the samples were incubated at 60° for 2 hours, 80° C. for 20 minutes, then 4° C. The quantity of each sample was then measured.

Removal of residual linear DNA by Exonuclease digestion. First, 30 μl of each Ligase sample was added to a nuclease-free PCR tube, then 3 μl water, 4 μl 10× Exonuclease Reaction Buffer (New England Biolabs Part No. B0293S), 1.5 μl Exonuclease I (20 U/μl, New England Biolabs Part No. M0293L), and 1.5 μl Exonuclease III (100 U/μl, New England Biolabs Part No. M0206L) were added to each sample. The samples were incubated at 37° C. for 45 minutes. Next, 75 mM EDTA, ph 8.0 was added to each sample and the samples were incubated at 85° C. for 5 minutes, then brought down to 4° C. The samples were then transferred to clean nuclease-free tubes. Next, 500 μl of PN buffer (supplied with QIAprep PCR Purification Kits) was added to each tube, mixed and the samples were applied to QIAprep Miniprep columns (Qiagen Part No. 28106) in 2 ml collection tubes and centrifuged for 1 minute at 14,000 rpm. The flow through was discarded, and 0.75 ml PE buffer (supplied with QIAprep PCR Purification Kits) was added to each column, and the column was centrifuged for an additional 1 minute. Again the flow through was discarded. The column was transferred to a fresh tube and 40 μl of EB buffer (supplied with QIAprep PCR Purification Kits) was added. The columns were spun at 14,000 for 1 minute to elute the single-stranded library constructs. The quantity of each sample was then measured.

Circle dependent replication for amplicon production: 40 fmol of exonuclease-treated single-stranded circles were added to nuclease-free PCR strip tubes, and water was added to bring the final volume to 10.0. μl. Next, 20 μl of phi 29 Mix (14 μl water, 2 μl 10× phi29 Reaction Buffer (New England Biolabs Part No. B0269S), 3.2 dNTP mix (2.5 mM of each dATP, dCTP, dGTP and dTTP), and 0.8 μl phi29 DNA polymerase (10 U/μl, New England Biolabs Part No. M0269S) was added to each tube. The tubes were then incubated at 30° C. for 120 minutes. The tubes were then removed, and 75 mM EDTA, pH 8.0 was added to each sample. The quantity of circle dependent replication product was then measured.

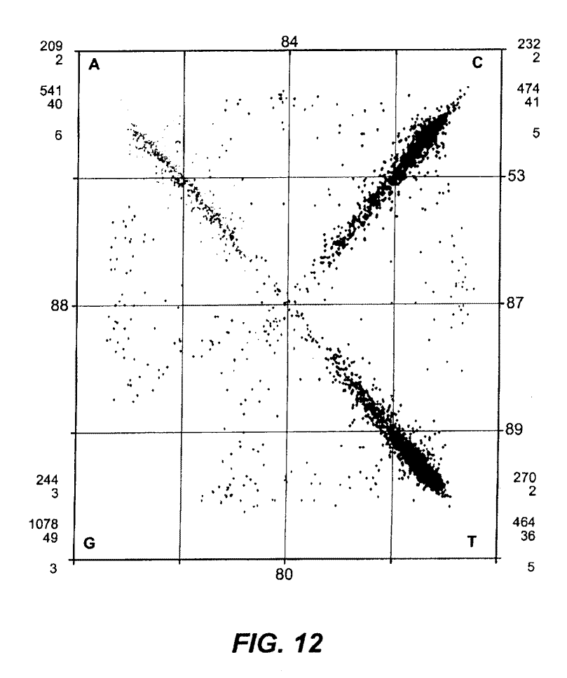

Determining amplicon quantity: The efficiency of the amplicon production for each construct was determined in the same reaction conditions (described above) with the initial constructs provided in a 1:1:1:1 ratio, which should result in an approximately equal distribution of the number of amplicons produced. Each of the four adaptor recognition sequences used in the exemplary assay was complementary to a specific probe labeled with a fluorophore detectable as a specific color: blue, red, yellow or green. In this example, each of the four recognition sequences of the individual adaptors comprises a different nucleotide from the other three, both at the 5′ end of the recognition sequence and at the 3′ end of the recognition sequence. Amplicon production was measured by plotting the occurrence of each detected hybridization, as illustrated in FIG. 12, and the measurements were used, both individual population measurements and ratios between the different populations, to determine the overall quantity and the relative percentage of each of the amplicon populations.

Determining amplicon quality: Once the quantity of the amplicons was determined, the quality of the amplicons was assessed by looking at color purity. The amplicons were suspended in amplicon dilution buffer (0.8× phi29 Reaction Buffer (New England Biolabs Part No. B0269S) and 10 mM EDTA, pH 8.0), and various dilutions were added into lanes of a flowslide and incubated at 30° C. for 30 minutes. The flowslides were then washed with buffer and a probe solution containing four different 12-mer probes labeled with either Cy5, Texas Red, FITC or Cy3 was added to each lane. The flowslides were transferred to a hot block pre-heated to 30° C. and incubated at 30° C. for 30 minutes. The flowslides were then imaged using Imager 3.2.1.0 software.

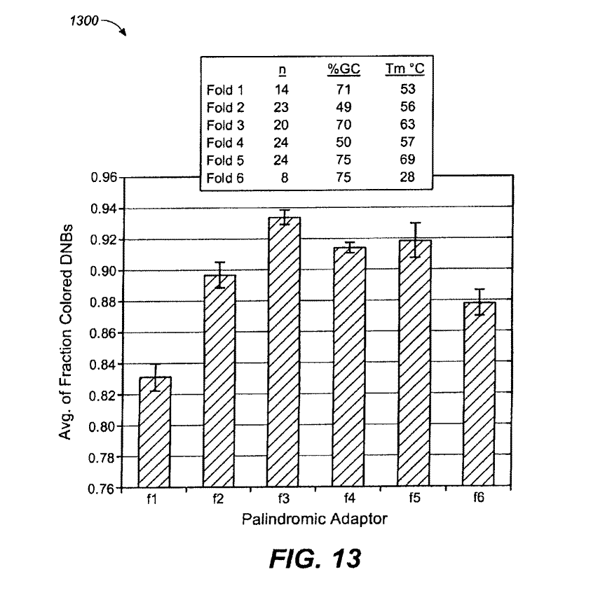

FIG. 13 is a chart showing characteristics of exemplary test stabilizing sequences in amplicons, with stabilizing sequences ranging in size from 8 to 24 nucleotides. The total number of nucleotides (“n”), percentage GC content and Tₘ are shown for each. The following sequences were tested using these methods:

(SEQ ID NO. 10)

f1: AGACAAGCTCGAGCTCGAGCGA

(SEQ ID NO. 11)

f2: AGACAACAAGATCGAGCTCGATCTTGACTCCTG

(SEQ ID NO. 12)

f3: AGACAACACGGTCGAGCTCGACCGTGACTCCTG

(SEQ ID NO. 13)

f4: AGACAACAGAAGATCGAGCTCGATCTTCTGACTCCTG

(SEQ ID NO. 14)

f5: AGACAACCGACGGTCGAGCTCGACCGTCGGACTCCTG

(SEQ ID NO. 15)

f6: AGACAACGAGCTGCACTCCTG

The graph in FIG. 13 shows the average fraction of color purity of amplicons containing adaptors with these exemplary stabilizing sequences. Note that the percentage of color purity ranges from a low of about 83% to a high of about 93%. The performance of each stabilizing sequence was found to vary, and the length of the stabilizing sequence, the CG content of the sequence, and the Tₘ may all contribute to this.

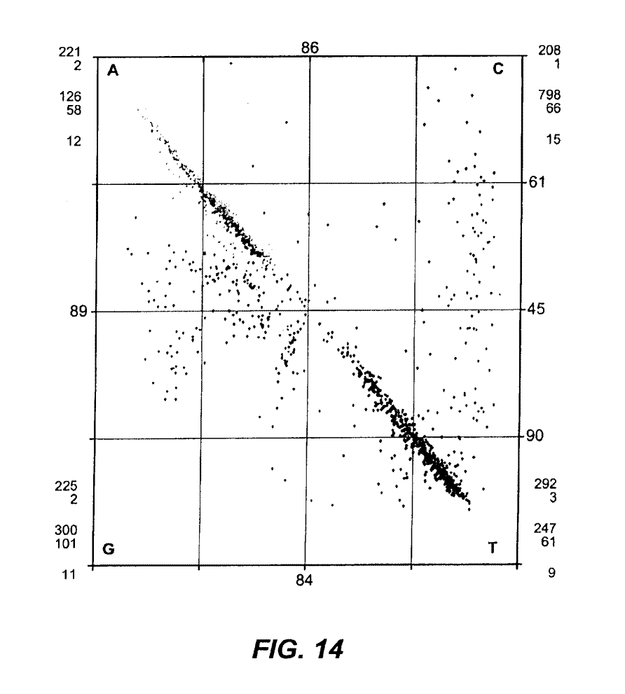

Repeat Element Model System: Efficiency of amplicon production for constructs having probe binding regions placed upstream of poly-nucleotide repeats was also determined, and definitive differences in amplicon production were shown using the various sequences. A 12 nucleotide repeat of A, T, G or C was placed at a pre-determined distance from the 3′ end of the probe hybridization sequence of an adaptor within a single sequencing construct, to provide one construct template with each poly-nucleotide repeat. The four construct populations were subject to CDR using phi29 as described above, and amplicon production was measured by plotting the occurrence of each detected hybridization event. The individual population measurements and ratios between the different populations were used to determine the overall quantity and the relative percentage of each of the amplicon populations, and the amplicon populations produced from each of the four constructs was measured and plotted as shown in FIG. 14.

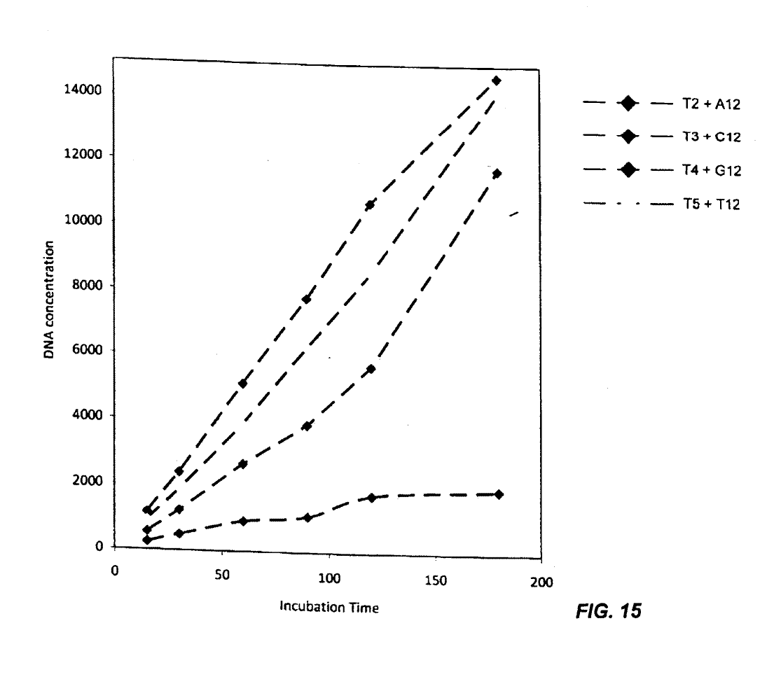

The construct containing the poly-G repeats was under-represented in this population, suggesting that the polymerization of the amplicons was inefficient using this particular adaptor in this sequence context under certain conditions. The production bias against this particular construct was confirmed through a measurement of the amplicons produced for each population as a function of time. Using 250 pM starting concentration for each adaptor-comprising construct in a phi29 replication reaction as described in the preceding example, the level of the poly-G amplicons is far lower as compared to the other three poly-nucleotide amplicon populations produced over time (FIG. 15). This illustrates the identification of sequences within an adaptor that do not promote efficient polymerization in the presence of a specific sequence within target nucleic acid sequences. Thus, the use of specific sequences within an adaptor may be eliminated, or alternatively different sequences may be used within a single amplicon polymerization reaction to prevent bias against the creation of amplicons that contain a sequence known to have certain limitations.

Inhibition of intermolecular amplicon interactions in amplicon libraries: The use of palindromic sequences in adaptors was also shown to reduce the molecular interactions between amplicons produced as described above. The model system as illustrated in FIG. 9 was used to assess the effect of different palindromic sequences on amplicon representation. Three sets of template constructs, each having adaptors comprising a palindromic sequence (f1, f3 or f5) interspersed with unknown target nucleic acid fragments, were used to examine the difference of the palindromes on amplicon interaction and sequencing efficiency. 750 picomol from each of the three sets of template constructs were used to create amplicon populations. The two palindromic sequences with higher predicted Tₘs, f3 and f5, displayed an approximately five-fold improvement in inhibiting amplicon interactions and increasing amplicon sequence representation as compared with use of the f1 palindrome, which has a Tₘ between 10 degrees less that f3 and 16 degrees less than f5.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

What is claimed:

1. A method for synthesizing nucleic acid amplicons with enhanced stability, said method comprising:
(a) providing a target nucleic acid;
(b) ligating a first arm of a first adaptor to one end of said target nucleic acid and a second arm of said first adaptor to the other end of said target nucleic acid, to form a first linear construct, wherein said first adaptor further comprises a recognition site for a first type IIs restriction endonuclease;
(c) amplifying said first linear construct with primers comprising one or more stabilizing sequences to produce amplification products;
(d) circularizing said amplification products to form circular templates;
(e) amplifying said circular templates using a rolling circle replication method to form said nucleic acid amplicons.

2. The method of claim 1, wherein said amplification products are double-stranded and wherein prior to circularizing step (d), the strands of said double-stranded amplification products are separated to produce single-stranded amplification products.

3. The method of claim 1, wherein prior to said amplifying step (e), said method further comprises:
(a) cleaving said circular templates with said first type IIs restriction endonucleases to form second linear constructs;
(b) ligating a first arm of a second adaptor to one end of said second linear constructs and a second arm of said second adaptor to the other end of said second linear constructs to form third linear constructs;
(c) circularizing said third linear constructs to form circular templates.

4. The method of claim 3, wherein said steps (a) through (c) are repeated until a desired number of adaptors are added.

5. The method of claim 3, wherein prior to said circularizing step (c), said method further comprises amplifying said third linear constructs with primers comprising one or more stabilizing sequences to produce amplified third linear constructs.

6. The method of claim 1, wherein said second adaptor comprises a recognition site for a second type IIs restriction endonuclease.

7. The method of claim 1, wherein said first adaptor further comprises a recognition site for a Type III restriction endonuclease.

8. The method of claim 1, wherein said rolling circle replication method is initiated at two different points on said circular templates simultaneously such that two concatemeric strands are produced.

9. The method of claim 1, wherein said first adaptor further comprises an anchor site.

10. The method of claim 9, wherein said anchor site does not overlap with said one or more stabilizing sequences.

11. The method of claim 1, wherein said at least one of said one or more stabilizing sequences comprises a palindrome.

12. The method of claim 11, wherein said palindrome has a nucleotide sequence according to SEQ ID NO: 5.

13. The method of claim 1, wherein said target nucleic acid in providing step (a) is a construct comprising a target sequence and an adaptor.

14. A composition comprising a nucleotide sequence according to SEQ ID NO: 1.

15. A composition comprising a nucleotide sequence according to SEQ ID NO: 2.

16. A composition comprising a nucleotide sequence according to SEQ ID NO: 3.

17. A composition comprising a nucleotide sequence according to SEQ ID NO: 4.

18. The composition of claim 14, wherein said composition further comprises a nucleotide sequence according to SEQ ID NO: 2.

19. The composition of claim 18, wherein said composition further comprises a nucleotide sequence according to SEQ ID NO: 3.

20. The composition of claim 19, wherein said composition further comprises a nucleotide sequence according to SEQ ID NO: 4.

21. The composition of claim 18, wherein said composition further comprises a first target sequence adjacent to said nucleotide sequence according to SEQ ID NO: 1 and a second target sequence adjacent to said nucleotide sequence according to SEQ ID NO: 2.

22. The composition of claim 19, wherein said composition further comprises a first target sequence adjacent to said nucleotide sequence according to SEQ ID NO: 1, a second target sequence adjacent to said nucleotide sequence according to SEQ ID NO: 2, and a third target sequence adjacent to said nucleotide sequence according to SEQ ID NO: 3.

23. The composition of claim 20, wherein said composition further comprises a first target sequence adjacent to said nucleotide sequence according to SEQ ID NO: 1, a second target sequence adjacent to said nucleotide sequence according to SEQ ID NO: 2, a third target sequence adjacent to said nucleotide sequence according to SEQ ID NO: 3, and a fourth target sequence adjacent to said nucleotide sequence according to SEQ ID NO: 4.

24. The composition of claim 23, wherein said composition is a circular nucleic acid.

25. A concatemer comprising a plurality of monomers, wherein each of said monomers comprises a composition according to claim 23.

26. A substrate comprising a surface, wherein said surface comprises a plurality of concatemers, and wherein each of said concatemers is a concatemer according to claim 25.

27. A composition that comprises:
(a) a substrate, wherein said substrate comprises a surface; and
(b) a plurality of concatemers immobilized on said surface, wherein each monomer of each of said concatemers comprises:
i. a first adaptor that comprises a nucleotide sequence according to SEQ ID NO: 1;
ii. a second adaptor that comprises a nucleotide sequence according to SEQ ID NO: 2;
iii. a third adaptor that comprises a nucleotide sequence according to SEQ ID NO: 3;
iv. a fourth adaptor that comprises a nucleotide sequence according to SEQ ID NO: 4;
v. a first target sequence adjacent to said first adaptor;
vi. a second target sequence adjacent to said second adaptor;
vii. a third target sequence adjacent to said third adaptor; and
viii. a fourth target sequence adjacent to said fourth adaptor.

28. The composition of claim 27, wherein at least two of said first, second, third and fourth adaptors comprise a stabilization sequence, and wherein said stabilizing sequences promote intramolecular interaction of different monomers of said concatemer over intermolecular interaction between said concatemer and other concatemers.

29. The composition of claim 27, wherein at least one of said first, second, third and fourth adaptors further comprises an anchor site.
